SEQUENCE LISTING (234 sequences)

SEQ ID NO: 1 - DNA, 17743 bp, Artificial Sequence
  Description of Artificial Sequence Targeting vector for rosa26 locus expressing the wt tet-repressor
tagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgatccgtcgacgcccgggctagcttgcatgcctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccgatccgcctagcattcaaccatggctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcgtaaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattgttaattaaggccatagcggccgccctgaggccgcgggcgatcgcctaggggtaaccgaagttcctatactttctagagaataggaacttcggaataggaacttcttataatctagaagatctggatccacgattcgagggcccctgcaggtcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgccatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattcatatgaagttcctatactttctagagaataggaacttcggaataggaacttcaaaatgtcgcggcgcgccggtaaccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgc

SEQ ID NO: 2 - DNA, 17907 bp, Artificial Sequence
  Description of Artificial Sequence Targeting vector for rosa26 locus expressing the codon optimized tet- repressor
tagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgatccgtcgacgcccgggctagcttgcatgcctgcaggttttcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggcaattcaccatgtccagactggacaagagcaaagtcatcaactctgccttggagctcctgaatgaagttggcattgagggcttgaccaccaggaagctggcccagaagctgggtgtggagcagcctaccctgtactggcatgtgaagaacaagagggctctgcttgatgccctggccattgagatgttggacaggcaccacacccacttctgccctctggaaggggagtcctggcaggacttcctgaggaacaatgccaagagcttcagatgtgccttgctctcccaccgggatggtgccaaagttcacttgggcaccaggcctacagagaagcagtatgagaccctggagaaccagctggcattcctgtgccaacaaggcttctccctggaaaatgccttgtatgccctctctgctgtgggccacttcaccttgggctgtgtgctggaggaccaggagcaccaagttgccaaggaggagagggagacccccaccactgactccatgccaccactgctgcggcaagctattgagttgtttgaccaccaaggggctgagcctgcattcctttttggcctggaactgatcatctgtggcctggaaaagcagctgaaatgtgagtctggctctcacgtgcccaaaaagagaaagcacgtgcctgctgatgccttggatgattttgacctggacatgctgcctgctgatgccctggatgactttgatttggacatgctccctgctgatgcacttgatgattttgacctggatatgctgtgatggatccatcgattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgcccccgggtaccgagctcgaattgttaattaaggccatagcggccgccctgaggccgcgggcgatcgcctaggggtaaccgaagttcctatactttctagagaataggaacttcggaataggaacttcttataatctagaagatctggatccacgattcgagggcccctgcaggtcaattctaccgggtaggggaggcgcttttcccaaggcagtctggagcatgcgctttagcagccccgctggcacttggcgctacacaagtggcctctggcctcgcacacattccacatccaccggtagcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcgaaggtcctcccgaggcccggcattctcgcacgcttcaaaagcgcacgtctgccgcgctgttctcctcttcctcatctccgggcctttcgacgatccagccgccaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgccatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtcgatgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcctgcaggaattcatatgaagttcctatactttctagagaataggaacttcggaataggaacttcaaaatgtcgcggcgcgccggtaaccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacgc

SEQ ID NO: 3 - DNA, 16467 bp, Artificial Sequence
  Description of Artificial Sequence Targeting vector for rosa26 locus containing the H1-tet inducible shRNA
ttacgctagggataacagggtaatatagccgcggtggcggccgctctagaactagtggatcccccgggctgcaggaattcgatatcaagcttatcgataccgtcatcgcaaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgagcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccgtcgacttaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagtccctatcagtgatagagattcccaggattccaattcagcgggagccacctgatgaagcttgatcgggtggctctcgctgagttggaatccatttttttctaggataacttcgtataatgtatgctatacgaagttatggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagag

SEQ ID NO: 4 - DNA, 23 bp, Artificial Sequence
  Description of Artificial Sequence Primer Rscreen1s
gacaggacagtgcttgtttaagg

SEQ ID NO: 5 - DNA, 23 bp, Artificial Sequence
  Description of Artificial Sequence Primer Rscreen1as
tgactacacaatattgctcgcac

SEQ ID NO: 6 - DNA, 1073 bp, Artificial Sequence
  Description of Artificial Sequence 5′ arm for Rosa26
caggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtct

SEQ ID NO: 7 - DNA, 4333 bp, Artificial Sequence
  Description of Artificial Sequence 3′ arm for Rosa26
tagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatc

SEQ ID NO: 8 - DNA, 75 bp, Artificial Sequence
  Description of Artificial Sequence Firefly luciferase-specific shRNA
aggattccaattcagcgggagccacctgatgaagcttgatcgggtggctctcgctgagttggaatccattttttt

SEQ ID NO: 9 - DNA, 25 bp, Artificial Sequence
  Description of Artificial Sequence Primer
atcgaattcaccatgtccagactgg

SEQ ID NO: 10 - DNA, 34 bp, Artificial Sequence
  Description of Artificial Sequence Primer
ataggatccttaagagccagactcacatttcagc

SEQ ID NO: 11 - DNA, 13139 bp, Artificial Sequence
  Description of Artificial Sequence Murine Rosa26 locus
aagcttctcacgtagcaaccagagctccagagccagcagctgctgccgccttgtatactcactcctgtgatccaacacaggagcaaccttttctttaccccacccccacttcttaacacacttttttttggggggggggggggaacaagtgctccatgctggaaggattggaactatgcttttagaaaggaacaatcctaaggtcacttttaaattgaggtctttgatttgaaaatcaacaaataccaaattccaaatattcgttttaattaaaccagcaatgtggatataagcattaagttttagttttaaaaaggtcaattttccaaacattcagcaatcatatttaaatttacagctaggaacaagagccttgggtcatgtcctaccaaagaacataactcaatattctacacatgacaatctgaataaccttaaagcctctaatcccataacaggccacaaattttggacagagaactaatgatcctcctgagaaaactggaagaaatccagggaaaagaaattcctgtgtcctccaaactcagaaatctctaattatgtcagtattctctgctttagtcctaggtcagattgcacacatctaaaataacctcttaaagttttcctcctagcgacctaaaccattattaatatcaaattaaccatcaaaacactttcctctcaatatgctgcacacaaacctcctcctggaacctcctccatctggatcctccccaatcaaaagtataggtatttaacatataagcaaggaagtaatgtaaacatgaccttggtcacaaatatgtcatctaaaaacaatttagtcaaggtatggaggaaattcgagaacctgaatctttttaagtattttgagcacaggaacaattggcaaaaggaatccaggtatagacaaaacccagagcccagagctctgggcgaaaaatgagttgctggtgaagacgttacacaagtaacatgagaaagcagaaaatgcaggtcatccacgcacccctgacccaggccagcagggcgggctgcagcatcagtacacaggagaaagatccttattcctaagaatgagaaaggcaaaggcgcccgatagaataaattagcatagaaggggctttcccaggagttaaaactttccttctgagcgattacctactaaaaccagggcttttgcccactaccatttacctaggatcttggcttgcacggattcataggggcatatccctccccctcttctttagagtcgttcttaaaagatcgctctccacgccctaggcagggaaaacgacaaaatctggctcaattccaggctagaaccctacaaattcaacagggatatcgcaaggatactggggcatacgccacagggagtccaagaatgtgaggtgggggtggcgaaggtaatgtctttggtgtgggaaaagcagcagccatctgagataggaactggaaaaccagaggagaggcgttcaggaagattatggaggggaggactgggcccccacgagcgaccagagttgtcacaaggccgcaagaacaggggaggtggggggctcagggacagaaaaaaaagtatgtgtattttgagagcagggttgggaggcctctcctgaaaagggtataaacgtggagtaggcaatacccaggcaaaaaggggagaccagagtagggggaggggaagagtcctgacccagggaagacattaaaaaggtagtggggtcgactagatgaaggagagcctttctctctgggcaagagcggtgcaatggtgtgtaaaggtagctgagaagacgaaaagggcaagcatcttcctgctaccaggctggggaggcccaggcccacgaccccgaggagagggaacgcagggagactgaggtgacccttctttcccccggggcccggtcgtgtggttcggtgtctcttttctgttggacccttaccttgacccaggcgctgccggggcctgggcccgggctgcggcgcacggcactcccgggaggcagcgagactcgagttaggcccaacgcggcgccacggcgtttcctggccgggaatggcccgtacccgtgaggtgggggtggggggcagaaaaggcggagcgagcccgagcggggagggggagggccaggggcggagggggccggcactactgtgttggcggactggcgggactagggctgcgtgagtctctgagcgcaggcgggcggcggccgcccctcccccggcggcggcagcggcggcagcggcggcagctcactcagcccgctgcccgagcggaaacgccactgaccgcacggggattcccagtgccggcgccaggggcacgcgggacacgccccctcccgccgcgccattggcctctccgcccaccgccccacacttattggccggtgcgccgccaatcagcggaggctgccggggccgcctaaagaagaggctgtgctttggggctccggctcctcagagagcctcggctaggtaggggatcgggactctggcgggagggcggcttggtgcgtttgcggggatgggcggccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagagtgtactataaggttgatggtaagcttataaggaacttgaaagtggagtaactactccatttctctgaggggagaattaaaatttttgaccaagtgttgttgagccactgagaatggtctcagaacataacttcttaaggaaccttcccagattgccctcaacactgcaccacatttggtcctgcttgaacattgccatggctcttaaagtcttaattaagaatattaattgtgtaattattgtttttcctcctttagatcattccttgaggacaggacagtgcttgtttaaggctatatttctgctgtctgagcagcaacaggtcttcgagatcaacatgatgttcataatcccaagatgttgccatttatgttctcagaagcaagcagaggcatgatggtcagtgacagtaatgtcactgtgttaaatgttgctatgcagtttggatttttctaatgtagtgtaggtagaacatatgtgttctgtatgaattaaactcttaagttacaccttgtataatccatgcaatgtgttatgcaattaccattttaagtattgtagctttctttgtatgtgaggataaaggtgtttgtcataaaatgttttgaacatttccccaaagttccaaattataaaaccacaacgttagaacttatttatgaacaatggttgtagtttcatgcttttaaaatgcttaattattcaattaacaccgtttgtgttataatatatataaaactgacatgtagaagtgtttgtccagaacatttcttaaatgtatactgtctttagagagtttaatatagcatgtcttttgcaacatactaacttttgtgttggtgcgagcaatattgtgtagtcattttgaaaggagtcatttcaatgagtgtcagattgttttgaatgttattgaacattttaaatgcagacttgttcgtgttttagaaagcaaaactgtcagaagctttgaactagaaattaaaaagctgaagtatttcagaagggaaataagctacttgctgtattagttgaaggaaagtgtaatagcttagaaaatttaaaaccatatagttgtcattgctgaatatctggcagatgaaaagaaatactcagtggttcttttgagcaatataacagcttgttatattaaaaattttccccacagatataaactctaatctataactcataaatgttacaaatggatgaagcttacaaatgtggcttgacttgtcactgtgcttgttttagttatgtgaaagtttggcaataaacctatgtcctaaatagtcaaactgtggaatgactttttaatctattggtttgtctagaacagttatgttgccatttgccctaatggtgaaagaaaaagtggggagtgccttggcactgttcatttgtggtgtgaaccaaagaggggggcatgcacttacacttcaaacatccttttgaaagactgacaagtttgggtcttcacagttggaattgggcatcccttttgtcagggagggagggagggagggaggctggcttgttatgctgacaagtgtgattaaattcaaactttgaggtaagttggaggaacttgtacattgttaggagtgtgacaatttggactcttaatgatttggtcatacaaaatgaacctagaccaacttctggaagatgtatataataactccatgttacattgatttcacctgactaatacttatcccttatcaattaaatacagaagatgccagccatctgggccttttaacccagaaatttagtttcaaactcctaggttagtgttctcactgagctacatcctgatctagtcctgaaaataggaccaccatcacccccaaaaaaatctcaaataagatttatgctagtgtttcaaaattttaggaataggtaagattagaaagttttaaattttgagaaatggcttctctagaaagatgtacatagtgaacactgaatggctcctaaagagcctagaaaactggtactgagcacacaggactgagaggtctttcttgaaaagcatgtattgctttacgtgggtcacagaaggcaggcaggaagaacttgggctgaaactggtgtcttaagtggctaacatcttcacaactgatgagcaagaactttatcctgatgcaaaaaccatccaaacaaactaagtgaaaggtggcaatggatcccaggctgctctagaggaggacttgacttctcatcccatcacccacaccagatagctcatagactgccaattaacaccagcttctagcctccacaggcacctgcactggtacacataatttcacacaaacacagtaagaagccttccacctggcatggtattgcttatctttagttcccaacacttgggaggcagaggccagccagggctatgtgacaaaaaccttgtctagaggagaaacttcatagcttatttcctattcacgtaaccaggttagcaaaatttaccagccagagatgaagctaacagtgtccactatatttgtagtgttttaagtcaattttttaaatatacttaatagaattaaagctatggtgaaccaagtacaaacctggtgtattaacttgagaacttagcataaaaagtagttcatttgttcagtaaatattaaatgcttactggcaaagattatgtcaggaacttggtaaatggtgatgaaacaatcatagttgtacatcttggttctgtgatcaccttggtttgaggtaaaagtggttcctttgatcaaggatggaattttaagtttatattcaatcaataatgtattattttgtgattgcaaaattgcctatctagggtataaaacctttaaaaatttcataataccagttcattctccagttactaattccaaaaagccactgactatggtgccaatgtggattctgttctcaaaggaaggattgtctgtgccctttattctaatagaaacatcacactgaaaatctaagctgaaagaagccagactttcctaaataaataactttccataaagctcaaacaaggattacttttaggaggcactgttaaggaactgataagtaatgaggttacttatataatgatagtcccacaagactatctgaggaaaaatcagtacaactcgaaaacagaacaaccagctaggcaggaataacagggctcccaagtcaggaggtctatccaacacccttttctgttgagggccccagacctacatattgtatacaaacagggaggtgggtgattttaactctcctgaggtaccttggtaaatctttgtcctgagtaagcagtacagtgtacagtttacattttcatttaaagatacattagctccctctaccccctaagactgacaggcactttgggggtggggagggctttggaaaataacgcttccatacactaaaagagaaatttctttaattaggcttgttggttccatacatctactggtgtttctactacttagtaatattataatagtcacacaagcatctttgctctgtttaggttgtatatttattttaaggcagatgataaaactgtagatcttaagggatgcttctgcttctgagatgatacaaagaatttagaccataaaacagtaggttgcacaagcaatagaatatggcctaaagtgttctgacacttagaagccaagcagtgtaggcttcttaagaaataccattacaatcaccttgctagaaatcaagcattctggagtggtcaagcagtgtaacctgtactgtaagttacttttctgctatttttctcccaaagcaagttctttatgctgatatttccagtgttaggaactacaaatattaataagttgtcttcactcttttctttaccaaggagggtctcttccttcatcttgatctgaaggatgaacaaaggcttgagcagtgcgctttagaagataaactgcagcatgaaggcccccgatgttcacccagactacatggacctttcgccacacatgtcccattccagataaggcctggcacacacaaaaaacataagtcattaggctaccagtctgattctaaaacaacctaaaatcttcccacttaaatgctatgggtggtgggttggaaagttgactcagaaaatcacttgctgtttttagagaggatctgggttcagtttctgatacattgtggcttacaactataactccagttctagggggtccatccaacatcctcttctgttgagggcaccaaataaatgtattgtgtacaaacagggaggtgagtgatttaactctcgtgtatagtaccttggtaaaacatttcttgtcctgagtaagcagtacagctctgcctgtccctggtctacagacacggctcatttcccgaaggcaagctggatagagattccaatttctcttcttggatcccatcctataaaagaaggtcaagtttaatctattgcaaaaggtaaataggtagtttcttacatgagacaagaacaaatcttaggtgtgaagcagtcatcttttacaggccagagcctctattctatgccaatgaaggaaactgttagtccagtgttatagagttagtccagtgtatagttttctatcagaacactttttttttaaacaactgcaacttagcttattgaagacaaaccacgagtagaaatctgtccaagaagcaagtgcttctcagcctacaatgtggaataggaccatgtaatggtacagtgagtgaaatgaattatggcatgtttttctgactgagaagacagtacaataaaaggtaaactcatggtatttatttaaaaagaatccaatttctacctttttccaaatggcatatctgttacaataatatccacagaagcagttctcagtgggaggttgcagatatcccactgaacagcatcaatgggcaaaccccaggttgtttttctgtggagacaaaggtaagatatttcaatatattttcccaagctaatgagatggctcagcaaataatggtactggccattaagtctcatgacctgagcttgatcctcagggaccatgtggtacaaggagagacctaaatccttcagttggacttcaatcttctaccctcatgtccacacacaaataaatacaataaaaaacattctgcagtcgaatttctaaaagggcgaat

SEQ ID NO: 12 - DNA, 21 bp, Artificial Sequence
  Description of Artificial Sequence DNA encoding siRNA 1
gcctgtgcctcttcagctacc

SEQ ID NO: 13 - DNA, 21 bp, Artificial Sequence
  Description of Artificial Sequence DNA encoding siRNA 2
gcggagacagcgacgaagagc

SEQ ID NO: 14 - DNA, 19 bp, Artificial Sequence
  Description of Artificial Sequence DNA encoding siRNA 3
cttattggagagagcacga

SEQ ID NO: 15 - DNA, 30 bp, Artificial Sequence
  Description of Artificial Sequence Primer 1 for isolation of SA from adenovirus
atacctgcaggggtgacctgcacgtctagg

SEQ ID NO: 16 - DNA, 32 bp, Artificial Sequence
  Description of Artificial Sequence Primer 2 for isolation of SA from adenovirus
atacctgcaggagtactggaaagaccgcgaag

SEQ ID NO: 17 - DNA, 35 bp, Artificial Sequence
  Description of Artificial Sequence Primer 1 for isolation of H1 promoter
aactatggccggccgaagaactcgtcaagaaggcg

SEQ ID NO: 18 - DNA, 39 bp, Artificial Sequence
  Description of Artificial Sequence Primer 2 for isolation of H1 promoter
tatggtaccgtttaaacgcggccgcaaatttattagagc

SEQ ID NO: 19 - DNA, 47 bp, Artificial Sequence
  Description of Artificial Sequence DNA encoding an shRNA directed against the CDH-1 gene
tgagaagtctcccagtcagttcaagagactgactgggagacttctca

SEQ ID NO: 20 - DNA, 47 bp, Artificial Sequence
  Description of Artificial Sequence DNA encoding an shRNA directed against the p53 gene
gactccagtggtaatctacttcaagagagtagattaccactggagtc

SEQ ID NO: 21 - DNA, 47 bp, Artificial Sequence
  Description of Artificial Sequence DNA encoding an shRNA directed against the CDC20 gene
cggcaggactccgggccgattcaagagatcggcccggagtcctgccg

SEQ ID NO: 22 - DNA, 47 bp, Artificial Sequence
  Description of Artificial Sequence DNA encoding an shRNA directed against the CYLD gene
cctcatgcagttctctttgttcaagagacaaagagaactgcatgagg

SEQ ID NO: 23 - DNA, 50 bp, Artificial Sequence
  Description of Artificial Sequence DNA encoding an shRNA directed against the RAS-GAP gene
aagatgaagccactccctatttcaagagaaaatagggagtggcttcatct

SEQ ID NO: 24 - DNA, 41 bp, Artificial Sequence
  Description of Artificial Sequence DNA encoding an shRNA directed against the tubulin gene
gacagagccaagtggactcacagagtccacttggctctgtc

SEQ ID NO: 25 - DNA, 42 bp, Artificial Sequence
  Description of Artificial Sequence DNA encoding an shRNA directed against the lamin gene
ctggacttccagaagaacattcgtgttcttctggaagtccag

SEQ ID NO: 26 - DNA, 47 bp, Artificial Sequence
  Description of Artificial Sequence DNA encoding an shRNA directed against the gene for the ubiquitin carboxyl-terminal hydrolase 12
gagattggtccagaacagtttcaagagaactgttctggaccaatctc

SEQ ID NO: 27 - DNA, 47 bp, Artificial Sequence
  Description of Artificial Sequence DNA encoding an shRNA directed against the gene for  the ubiquitin carboxyl-terminal hydrolase 12
gcccttccgatcatggtagttcaagagactaccatgatcggaagggc

SEQ ID NO: 28 - DNA, 47 bp, Artificial Sequence
  Description of Artificial Sequence DNA encoding an shRNA directed against the gene for the ubiquitin carboxyl-terminal hydrolase 12
tctttagaattcttaagtattcaagagatacttaagaattctaaaga

SEQ ID NO: 29 - DNA, 47 bp, Artificial Sequence
  Description of Artificial Sequence DNA encoding an shRNA directed against the gene of the ubiquitin carboxyl-terminal hydrolase
cattagctatatcaacatgttcaagagacatgttgatatagctaatg

SEQ ID NO: 30 - DNA, 47 bp, Artificial Sequence
  Description of Artificial Sequence DNA encoding an shRNA directed against the gene for the ubiquitin carboxyl-terminal hydrolase11
accacaaacggcggaacgattcaagagatcgttccgccgtttgtggt

SEQ ID NO: 31 - DNA, 47 bp, Artificial Sequence
  Description of Artificial Sequence DNA encoding an shRNA directed against the gene for the ubiquitin carboxyl-terminal hydrolase 11
gagggtcttggaggtcttcttcaagagagaagacctccaagaccctc

SEQ ID NO: 32 - DNA, 47 bp, Artificial Sequence
  Description of Artificial Sequence DNA encoding an shRNA directed against the gene for the ubiquitin carboxyl-terminal hydrolase 11
gtccatgcccagccgtacattcaagagatgtacggctgggcatggac

SEQ ID NO: 33 - DNA, 47 bp, Artificial Sequence
  Description of Artificial Sequence DNA encoding an shRNA directed against the gene for the ubiquitin carboxyl-terminal hydrolase 11
gctggacaccctcgtggagttcaagagactccacgagggtgtccagc

SEQ ID NO: 34 - DNA, 47 bp, Artificial Sequence
  Description of Artificial Sequence DNA encoding an shRNA directed against the gene for the ubiquitin carboxyl-terminal hydrolase 10
gaatatcagagaattgagtttcaagagaactcaattctctgatattc

SEQ ID NO: 35 - DNA, 47 bp, Artificial Sequence
  Description of Artificial Sequence DNA encoding an shRNA directed against the gene for the ubiquitin carboxyl-terminal hydrolase 10
tggacttcatgaggaaatgttcaagagacatttcctcatgaagtcca

SEQ ID NO: 36 - DNA, 47 bp, Artificial Sequence
  Description of Artificial Sequence DNA encoding an shRNA directed against the  gene for the ubiquitin carboxyl-terminal hydrolase 10
tattgaatatcctgtggacttcaagagagtccacaggatattcaata

SEQ ID NO: 37 - DNA, 47 bp, Artificial Sequence
  Description of Artificial Sequence DNA encoding an shRNA directed against the gene for the ubiquitin carboxyl-terminal hydrolase 10
ttgtactgagagaaactgcttcaagagagcagtttctctcagtacaa

SEQ ID NO: 38 - DNA, 47 bp, Artificial Sequence
  Description of Artificial Sequence DNA encoding an shRNA directed against the HAUSP gene
gatcaatgataggtttgaattcaagagattcaaacctatcattgatc

SEQ ID NO: 39 - DNA, 47 bp, Artificial Sequence
  Description of Artificial Sequence DNA encoding an shRNA directed against the HAUSP gene
ggagtttgagaagtttaaattcaagagatttaaacttctcaaactcc

SEQ ID NO: 40 - DNA, 47 bp, Artificial Sequence
  Description of Artificial Sequence DNA encoding an shRNA directed against the HAUSP gene
gaactcctcgcttgctgagttcaagagactcagcaagcgaggagttc

SEQ ID NO: 41 - DNA, 47 bp, Artificial Sequence
  Description of Artificial Sequence DNA encoding an shRNA directed against the HAUSP gene
ccgaatttaacagagagaattcaagagattctctctgttaaattcgg

SEQ ID NO: 42 - DNA, 47 bp, Artificial Sequence
  Description of Artificial Sequence DNA encoding an shRNA directed against the gene for the ubiquitin carboxyl-terminal hydrolase 8
gacagcagaagaatgcagattcaagagatctgcattcttctgctgtc

SEQ ID NO: 43 - DNA, 47 bp, Artificial Sequence
  Description of Artificial Sequence DNA encoding an shRNA directed against the gene for the ubiquitin carboxyl-terminal hydrolase 8
ataaagctcaacgagaaccttcaagagaggttctcgttgagctttat

SEQ ID NO: 44 - DNA, 47 bp, Artificial Sequence
  Description of Artificial Sequence DNA encoding an shRNA directed against the gene for the ubiquitin carboxyl-terminal hydrolase 8
ggtgaagtggcagaagaatttcaagagaattcttctgccacttcacc

SEQ ID NO: 45 - DNA, 47 bp, Artificial Sequence
  Description of Artificial Sequence DNA encoding an shRNA directed against the gene for the ubiquitin carboxyl-terminal hydrolase 8
gtattgcagtaatcatcacttcaagagagtgatgattactgcaatac

SEQ ID NO: 46 - DNA, 47 bp, Artificial Sequence
  Description of Artificial Sequence DNA encoding an shRNA directed against the FLJ10785 gene
gatatggggttccatgtcattcaagagatgacatggaaccccatatc

SEQ ID NO: 47 - DNA, 47 bp, Artificial Sequence
  Description of Artificial Sequence DNA encoding an shRNA directed against the FLJ10785 gene
ggagacatggttcttagtgttcaagagacactaagaaccatgtctcc

SEQ ID NO: 48 - DNA, 47 bp, Artificial Sequence
  Description of Artificial Sequence DNA encoding an shRNA directed against the FLJ10785 gene
agcaccaagttcgtctcagttcaagagactgagacgaacttggtgct

SEQ ID NO: 49 - DNA, 47 bp, Artificial Sequence
  Description of Artificial Sequence DNA encoding an shRNA directed against the FLJ10785 gene
gatgcaacactgaaagaacttcaagagagttctttcagtgttgcatc

SEQ ID NO: 50 - DNA, 47 bp, Artificial Sequence
  Description of Artificial Sequence DNA encoding an shRNA directed against the KIAA0710 gene
gtcaatggcagtgatgatattcaagagatatcatcactgccattgac

SEQ ID NO: 51 - DNA, 47 bp, Artificial Sequence
  Description of Artificial Sequence DNA encoding an shRNA directed against the KIAA0710 gene
cctgctagctgcctgtggcttcaagagagccacaggcagctagcagg

SEQ ID NO: 52 - DNA, 47 bp, Artificial Sequence
  Description of Artificial Sequence DNA encoding an shRNA directed against the KIAA0710 gene
ccacctttgccagaaggagttcaagagactccttctggcaaaggtgg

SEQ ID NO: 53 - DNA, 47 bp, Artificial Sequence
  Description of Artificial Sequence DNA encoding an shRNA directed against the KIAA0710 gene
ccctattgaggcaagtgtcttcaagagagacacttgcctcaataggg

SEQ ID NO: 54 - DNA, 47 bp, Artificial Sequence
  Description of Artificial Sequence DNA encoding an shRNA directed against the genes FLJ12552/FLJ14256
gaaggaaaacttgctgacgttcaagagacgtcagcaagttttccttc

SEQ ID NO: 55 - DNA, 47 bp, Artificial Sequence
  Description of Artificial Sequence DNA encoding an shRNA directed against the FLJ12552/FLJ14256 genes
ctcacctgggtccatgagattcaagagatctcatggacccaggtgag

SEQ ID NO: 56 - DNA, 47 bp, Artificial Sequence
  Description of Artificial Sequence DNA encoding an shRNA directed against the FLJ12552/FLJ14256 genes
gctgtcttaccgtgtggtcttcaagagagaccacacggtaagacagc

SEQ ID NO: 57 - DNA, 47 bp, Artificial Sequence
  Description of Artificial Sequence DNA encoding an shRNA directed against the FLJ12552/FLJ14256 genes
cctggaccgcatgtatgacttcaagagagtcatacatgcggtccagg

SEQ ID NO: 58 - DNA, 47 bp, Artificial Sequence; identical to SEQ ID NO: 50
  Description of Artificial Sequence DNA encoding an shRNA directed against the KIAA1203 gene

SEQ ID NO: 59 - DNA, 47 bp, Artificial Sequence; identical to SEQ ID NO: 51
  Description of Artificial Sequence DNA encoding an shRNA directed against the KIAA1203 gene

SEQ ID NO: 60 - DNA, 47 bp, Artificial Sequence; identical to SEQ ID NO: 52
  Description of Artificial Sequence DNA encoding an shRNA directed against the KIAA1203 gene

SEQ ID NO: 61 - DNA, 47 bp, Artificial Sequence; identical to SEQ ID NO: 53
  Description of Artificial Sequence DNA encoding an shRNA directed against the KIAA1203 gene

SEQ ID NO: 62 - DNA, 47 bp, Artificial Sequence
  Description of Artificial Sequence DNA encoding an shRNA directed against the FLJ23277 gene
ggaaatccgaattgcttggttcaagagaccaagcaattcggatttcc

SEQ ID NO: 63 - DNA, 47 bp, Artificial Sequence
  Description of Artificial Sequence DNA encoding an shRNA directed against the FLJ23277 gene
cacatttcttcaagtgtggttcaagagaccacacttgaagaaatgtg

SEQ ID NO: 64 - DNA, 47 bp, Artificial Sequence
  Description of Artificial Sequence DNA encoding an shRNA directed against the FLJ23277 gene
cagcaggatgctcaagaatttcaagagaattcttgagcatcctgctg

SEQ ID NO: 65 - DNA, 47 bp, Artificial Sequence
  Description of Artificial Sequence DNA encoding an shRNA directed against the FLJ23277 gene
gctgaatacctacattggcttcaagagagccaatgtaggtattcagc

SEQ ID NO: 66 - DNA, 47 bp, Artificial Sequence
  Description of Artificial Sequence DNA encoding an shRNA directed against the FLJ14914 (similar to UBP4) gene
gggcttgtgcctggccttgttcaagagacaaggccaggcacaagccc

SEQ ID NO: 67 - DNA, 47 bp, Artificial Sequence
  Description of Artificial Sequence DNA encoding an shRNA directed against the FLJ14914 (similar to UBP4) gene
gccttgtcctgccaagaagttcaagagacttcttggcaggacaaggc

SEQ ID NO: 68 - DNA, 47 bp, Artificial Sequence
  Description of Artificial Sequence DNA encoding an shRNA directed against the FLJ14914 (similar to UBP4) gene
gattgaagccaagggaacgttcaagagacgttcccttggcttcaatc

SEQ ID NO: 69 - DNA, 47 bp, Artificial Sequence
  Description of Artificial Sequence DNA encoding an shRNA directed against the FLJ14914 (similar to UBP4) gene
tggcgcctgctccccatctttcaagagaagatggggagcaggcgcca

SEQ ID NO: 70 - DNA, 47 bp, Artificial Sequence
  Description of Artificial Sequence DNA encoding an shRNA directed against the gene for the ubiquitin carboxyl-terminal hydrolase isozyme L5
gaaccagcaggctctgtggttcaagagaccacagagcctgctggttc

SEQ ID NO: 71 - DNA, 47 bp, Artificial Sequence
  Description of Artificial Sequence DNA encoding an shRNA directed against the gene for the ubiquitin carboxyl-terminal hydrolase isozyme L5
ggaagcataattatctgccttcaagagaggcagataattatgcttcc

SEQ ID NO: 72 - DNA, 47 bp, Artificial Sequence
  Description of Artificial Sequence DNA encoding an shRNA directed against the gene for the ubiquitin carboxyl-terminal hydrolase isozyme L5
agaagaagatgcttttcacttcaagagagtgaaaagcatcttcttct

SEQ ID NO: 73 - DNA, 47 bp, Artificial Sequence
  Description of Artificial Sequence DNA encoding an shRNA directed against the gene for the ubiquitin carboxyl-terminal hydrolase isozyme L5
cttgcagaggaggaacccattcaagagatgggttcctcctctgcaag

SEQ ID NO: 74 - DNA, 47 bp, Artificial Sequence
  Description of Artificial Sequence DNA encoding an shRNA directed against the gene for the ubiquitin carboxyl-terminal hydrolase isozyme L3
gcaaacaatcagcaatgccttcaagagaggcattgctgattgtttgc

SEQ ID NO: 75 - DNA, 47 bp, Artificial Sequence
  Description of Artificial Sequence DNA encoding an shRNA directed against the gene for the ubiquitin carboxyl-terminal hydrolase isozyme L3
ttggactgattcatgctatttcaagagaatagcatgaatcagtccaa

SEQ ID NO: 76 - DNA, 47 bp, Artificial Sequence
  Description of Artificial Sequence DNA encoding an shRNA directed against the gene for the ubiquitin carboxyl-terminal hydrolase isozyme L3
ctggcaattcgttgatgtattcaagagatacatcaacgaattgccag

SEQ ID NO: 77 - DNA, 47 bp, Artificial Sequence
  Description of Artificial Sequence DNA encoding an shRNA directed against the gene for the ubiquitin carboxyl-terminal hydrolase isozyme L3
ttagatgggcggaagccatttcaagagaatggcttccgcccatctaa

SEQ ID NO: 78 - DNA, 47 bp, Artificial Sequence
  Description of Artificial Sequence DNA encoding an shRNA directed against the gene for the ubiquitin carboxyl-terminal hydrolase isozyme L1
gaggagtctctgggctcggttcaagagaccgagcccagagactcctc

SEQ ID NO: 79 - DNA, 47 bp, Artificial Sequence
  Description of Artificial Sequence DNA encoding an shRNA directed against the gene for the ubiquitin carboxyl-terminal hydrolase isozyme L1
gagctgaagggacaagaagttcaagagacttcttgtcccttcagctc

SEQ ID NO: 80 - DNA, 47 bp, Artificial Sequence
  Description of Artificial Sequence DNA encoding an shRNA directed against the gene for the ubiquitin carboxyl-terminal hydrolase isozyme L1
tgtcgggtagatgacaaggttcaagagaccttgtcatctacccgaca

SEQ ID NO: 81 - DNA, 47 bp, Artificial Sequence
  Description of Artificial Sequence DNA encoding an shRNA directed against the gene for the ubiquitin carboxyl-terminal hydrolase isozyme L1
cacagctgttcttctgttcttcaagagagaacagaagaacagctgtg

SEQ ID NO: 82 - DNA, 47 bp, Artificial Sequence
  Description of Artificial Sequence DNA encoding an shRNA directed against the KIAA1891/FLJ25263 genes
gtggaagcctttacagatcttcaagagagatctgtaaaggcttccac

SEQ ID NO: 83 - DNA, 47 bp, Artificial Sequence
  Description of Artificial Sequence DNA encoding an shRNA directed against the KIAA1891/FLJ25236 genes
caacagctgccttcatctgttcaagagacagatgaaggcagctgttg

SEQ ID NO: 84 - DNA, 47 bp, Artificial Sequence
  Description of Artificial Sequence DNA encoding an shRNA directed against the KIAA1891/FLJ25263 genes
ccataggcagtcctcctaattcaagagattaggaggactgcctatgg

SEQ ID NO: 85 - DNA, 47 bp, Artificial Sequence
  Description of Artificial Sequence DNA encoding an shRNA directed against the KIAA1891/FLJ25263 genes
tgtatcactgccactggttttcaagagaaaccagtggcagtgataca

SEQ ID NO: 86 - DNA, 47 bp, Artificial Sequence
  Description of Artificial Sequence DNA encoding an shRNA directed against the FLJ14528 (similar to UBP8) gene
catgttgggcagctgcagcttcaagagagctgcagctgcccaacatg

SEQ ID NO: 87 - DNA, 47 bp, Artificial Sequence
  Description of Artificial Sequence DNA encoding an shRNA directed against the FLJ14528 (similar to UBP8) gene
cacaactggagacctgaagttcaagagacttcaggtctccagttgtg

SEQ ID NO: 88 - DNA, 47 bp, Artificial Sequence
  Description of Artificial Sequence DNA encoding an shRNA directed against the FLJ14528 (similar to UBP8) gene
gtatgcctccaagaaagagttcaagagactctttcttggaggcatac

SEQ ID NO: 89 - DNA, 47 bp, Artificial Sequence
  Description of Artificial Sequence DNA encoding an shRNA directed against the FLJ14528 (similar to UBP8) gene
cttcacagtacatttctctttcaagagaagagaaatgtactgtgaag

SEQ ID NO: 90 - DNA, 47 bp, Artificial Sequence
  Description of Artificial Sequence DNA encoding an shRNA directed against the gene for the U4/U6 TRI snRNP 65 kDa protein
gtactttcaaggccggggtttcaagagaaccccggccttgaaagtac

SEQ ID NO: 91 - DNA, 47 bp, Artificial Sequence
  Description of Artificial Sequence DNA encoding an shRNA directed against the gene for the U4/U6 TRI snRNP 65 kDa protein
cttggacaagcaagccaaattcaagagatttggcttgcttgtccaag

SEQ ID NO: 92 - DNA, 47 bp, Artificial Sequence
  Description of Artificial Sequence DNA encoding an shRNA directed against the gene for the U4/U6 TRI snRNP 65 kDa protein
gactattgtgactgatgttttcaagagaaacatcagtcacaatagtc

SEQ ID NO: 93 - DNA, 47 bp, Artificial Sequence
  Description of Artificial Sequence DNA encoding an shRNA directed against the gene for the U4/U6 TRI snRNP 65kDa protein
ggagaactttctgaagcgcttcaagagagcgcttcagaaagttctcc

SEQ ID NO: 94 - DNA, 47 bp, Artificial Sequence
  Description of Artificial Sequence DNA encoding an shRNA for the XM_089437 gene
gacgagagaaaccttcaccttcaagagaggtgaaggtttctctcgtc

SEQ ID NO: 95 - DNA, 47 bp, Artificial Sequence
  Description of Artificial Sequence DNA encoding an shRNA directed against the XM_089437 gene
acattattctacattctttttcaagagaaaagaatgtagaataatgt

SEQ ID NO: 96 - DNA, 47 bp, Artificial Sequence
  Description of Artificial Sequence DNA encoding an shRNA directed against the XM_089437 gene
agattcgcaaatggatgtattcaagagatacatccatttgcgaatct

SEQ ID NO: 97 - DNA, 47 bp, Artificial Sequence
  Description of Artificial Sequence DNA encoding an shRNA directed against the XM_089437 gene
cattcccaccatgagtctgttcaagagacagactcatggtgggaatg

SEQ ID NO: 98 - DNA, 47 bp, Artificial Sequence
  Description of Artificial Sequence DNA encoding an shRNA directed against the KIAA1453 gene
gatcgcccgacacttccgcttcaagagagcggaagtgtcgggcgatc

SEQ ID NO: 99 - DNA, 47 bp, Artificial Sequence
  Description of Artificial Sequence DNA encoding an shRNA directed against the KIAA1453 gene
ccagcaggcctacgtgctgttcaagagacagcacgtaggcctgctgg

SEQ ID NO: 100 - DNA, 47 bp, Artificial Sequence
  Description of Artificial Sequence DNA encoding an shRNA directed against the KIAA1453 gene
gccagctcctccacagcacttcaagagagtgctgtggaggagctggc

SEQ ID NO: 101 - DNA, 47 bp, Artificial Sequence
  Description of Artificial Sequence DNA encoding an shRNA directed against the KIAA1453 gene
cgccgccaagtggagcagattcaagagatctgctccacttggcggcg

SEQ ID NO: 102 - DNA, 47 bp, Artificial Sequence
  Description of Artificial Sequence DNA encoding an shRNA directed against the FLJ12697 gene
gaagatgcccatgaattccttcaagagaggaattcatgggcatcttc

SEQ ID NO: 103 - DNA, 47 bp, Artificial Sequence
  Description of Artificial Sequence DNA encoding an shRNA directed against the FLJ12697 gene
caaacaggctgcgccaggcttcaagagagcctggcgcagcctgtttg

SEQ ID NO: 104 - DNA, 47 bp, Artificial Sequence
  Description of Artificial Sequence DNA encoding an shRNA directed against the FLJ12697 gene
acggcctagcgcctgatggttcaagagaccatcaggcgctaggccgt

SEQ ID NO: 105 - DNA, 47 bp, Artificial Sequence
  Description of Artificial Sequence DNA encoding an shRNA directed against the FLJ12697 gene
ctgtaacctctctgatcggttcaagagaccgatcagagaggttacag

SEQ ID NO: 106 - DNA, 47 bp, Artificial Sequence
  Description of Artificial Sequence DNA encoding an shRNA directed against the gene for the ubiquitin specific protease (USP18)
tctgtcagtccatcctggcttcaagagagccaggatggactgacaga

SEQ ID NO: 107 - DNA, 47 bp, Artificial Sequence
  Description of Artificial Sequence DNA encoding an shRNA directed against the gene for the ubiquitin specific protease (USP18)
tgaagcgagagtcttgtgattcaagagatcacaagactctcgcttca

SEQ ID NO: 108 - DNA, 47 bp, Artificial Sequence
  Description of Artificial Sequence DNA encoding an shRNA directed against the gene for the ubiquitin specific protease (USP18)
gatggagtgctaatggaaattcaagagatttccattagcactccatc

SEQ ID NO: 109 - DNA, 47 bp, Artificial Sequence
  Description of Artificial Sequence DNA encoding an shRNA directed against the gene for the ubiquitin specific protease (USP18)
ccttcagagattgacacgcttcaagagagcgtgtcaatctctgaagg

SEQ ID NO: 110 - DNA, 47 bp, Artificial Sequence
  Description of Artificial Sequence DNA encoding an shRNA directed against the gene for the ubiquitin carboxyl-terminal hydrolase 20
cctgaccacgttccgactgttcaagagacagtcggaacgtggtcagg

SEQ ID NO: 111 - DNA, 47 bp, Artificial Sequence
  Description of Artificial Sequence DNA encoding an shRNA directed against the gene for the ubiquitin carboxyl-terminal hydrolase 20
gagttccttcgctgcctgattcaagagatcaggcagcgaaggaactc

SEQ ID NO: 112 - DNA, 47 bp, Artificial Sequence
  Description of Artificial Sequence DNA encoding an shRNA directed against the gene for the ubiquitin carboxyl-terminal hydrolase 20
gactgccttgctgccttctttcaagagaagaaggcagcaaggcagtc

SEQ ID NO: 113 - DNA, 47 bp, Artificial Sequence
  Description of Artificial Sequence DNA encoding an shRNA directed against the gene for the ubiquitin carboxyl-terminal hydrolase 20
cgccgagggctacgtactcttcaagagagagtacgtagccctcggcg

SEQ ID NO: 114 - DNA, 47 bp, Artificial Sequence
  Description of Artificial Sequence DNA encoding an shRNA directed against the gene for the ubiquitin carboxyl-terminal hydrolase 24
ggcgagaagaaaggactgtttcaagagaacagtcctttcttctcgcc

SEQ ID NO: 115 - DNA, 47 bp, Artificial Sequence
  Description of Artificial Sequence DNA encoding an shRNA directed against the gene for the ubiquitin carboxyl terminal hydrolase 24
ggacgagaattgataaagattcaagagatctttatcaattctcgtcc

SEQ ID NO: 116 - DNA, 47 bp, Artificial Sequence
  Description of Artificial Sequence DNA encoding an shRNA directed against the gene for the ubiquitin carboxyl-terminal hydrolase 24
gcacgagaatttgggaatcttcaagagagattcccaaattctcgtgc

SEQ ID NO: 117 - DNA, 47 bp, Artificial Sequence
  Description of Artificial Sequence DNA encoding an shRNA directed against the gene for the ubiquitin carboxyl-terminal hydrolase 24
ctacttcatgaaatattggttcaagagaccaatatttcatgaagtag

SEQ ID NO: 118 - DNA, 47 bp, Artificial Sequence
  Description of Artificial Sequence DNA encoding an shRNA directed against the KIAA1594 gene
gataacagcttcttgtctattcaagagatagacaagaagctgttatc

SEQ ID NO: 119 - DNA, 47 bp, Artificial Sequence
  Description of Artificial Sequence DNA encoding an shRNA directed against the KIAA1594 gene
gagaataggacatcagggcttcaagagagccctgatgtcctattctc

SEQ ID NO: 120 - DNA, 47 bp, Artificial Sequence
  Description of Artificial Sequence DNA encoding an shRNA directed against the KIAA1594 gene
cttggaagactgaacctgtttcaagagaacaggttcagtcttccaag

SEQ ID NO: 121 - DNA, 47 bp, Artificial Sequence
  Description of Artificial Sequence DNA encoding an shRNA directed against the KIAA1594 gene
caactcctttgtggatgcattcaagagatgcatccacaaaggagttg

SEQ ID NO: 122 - DNA, 47 bp, Artificial Sequence
  Description of Artificial Sequence DNA encoding an shRNA directed against the KIAA1350 gene
gatgttgtctccaaatgcattcaagagatgcatttggagacaacatc

SEQ ID NO: 123 - DNA, 47 bp, Artificial Sequence
  Description of Artificial Sequence DNA encoding an shRNA directed against the KIAA1350 gene
cgtggggactgtacctcccttcaagagagggaggtacagtccccacg

SEQ ID NO: 124 - DNA, 47 bp, Artificial Sequence
  Description of Artificial Sequence DNA encoding an shRNA direcetd against the KIAA1350 gene
gtacagcttcagaaccaagttcaagagacttggttctgaagctgtac

SEQ ID NO: 125 - DNA, 47 bp, Artificial Sequence
  Description of Artificial Sequence DNA encoding an shRNA directed against the gene for the ubiquitin carboxyl-terminal hydrolase 25
gatgatcttcagagagcaattcaagagattgctctctgaagatcatc

SEQ ID NO: 126 - DNA, 47 bp, Artificial Sequence
  Description of Artificial Sequence DNA encoding an shRNA directed against the gene for the ubiquitin carboxyl-terminal hydrolase 25
ggaacatcggaatttgcctttcaagagaaggcaaattccgatgttcc

SEQ ID NO: 127 - DNA, 47 bp, Artificial Sequence
  Description of Artificial Sequence DNA encoding an shRNA directed against the gene for the ubiquitin carboxyl-terminal hydrolase 25
gagctagtgagggactcttttcaagagaaagagtccctcactagctc

SEQ ID NO: 128 - DNA, 47 bp, Artificial Sequence
  Description of Artificial Sequence DNA encoding an shRNA directed against the gene for the ubiquitin carboxyl-terminal hydrolase 25
gcagggttctttaaggcaattcaagagattgccttaaagaaccctgc

SEQ ID NO: 129 - DNA, 47 bp, Artificial Sequence
  Description of Artificial Sequence DNA encoding an shRNA directed against the gene for the ubiquitin carboxyl-terminal hydrolase 16
tcgatgattcctctgaaacttcaagagagtttcagaggaatcatcga

SEQ ID NO: 130 - DNA, 47 bp, Artificial Sequence
  Description of Artificial Sequence DNA encoding an shRNA directed against the gene for the ubiquitin carboxyl-terminal hydrolase 16
gataatggaaatattgaacttcaagagagttcaatatttccattatc

SEQ ID NO: 131 - DNA, 47 bp, Artificial Sequence
  Description of Artificial Sequence DNA encoding an shRNA directed against the gene for the ubiquitin carboxyl-terminal hydrolase 16
gttcttcatttaaatgatattcaagagatatcatttaaatgaagaac

SEQ ID NO: 132 - DNA, 47 bp, Artificial Sequence
  Description of Artificial Sequence DNA encoding an shRNA directed against the gene for the ubiquitin carboxyl-terminal hydrolase 16
gttaacaaacacataaagtttcaagagaactttatgtgtttgttaac

SEQ ID NO: 133 - DNA, 47 bp, Artificial Sequence
  Description of Artificial Sequence DNA encoding an shRNA directed against the USP9X gene
gttagagaagattcttcgtttcaagagaacgaagaatcttctctaac

SEQ ID NO: 134 - DNA, 47 bp, Artificial Sequence
  Description of Artificial Sequence DNA encoding an shRNA directed against the USP9X gene
gttgattggacaattaaacttcaagagagtttaattgtccaatcaac

SEQ ID NO: 135 - DNA, 47 bp, Artificial Sequence
  Description of Artificial Sequence DNA encoding an shRNA directed against the USP9X gene
ggttgataccgtaaagcgcttcaagagagcgctttacggtatcaacc

SEQ ID NO: 136 - DNA, 47 bp, Artificial Sequence
  Description of Artificial Sequence DNA encoding an shRNA directed against the USP9X gene
gcaatgaaacgtccaatggttcaagagaccattggacgtttcattgc

SEQ ID NO: 137 - DNA, 47 bp, Artificial Sequence
  Description of Artificial Sequence DNA encoding an shRNA directed against the USP9Y gene
agctagagaaaattcttcgttcaagagacgaagaattttctctagct

SEQ ID NO: 138 - DNA, 47 bp, Artificial Sequence
  Description of Artificial Sequence DNA encoding an shRNA directed against the USP9Y gene
gatcctatatgatggatgattcaagagatcatccatcatataggatc

SEQ ID NO: 139 - DNA, 47 bp, Artificial Sequence
  Description of Artificial Sequence DNA encoding an shRNA directed against the USP9Y gene
gttcttcttgtcagtgaaattcaagagatttcactgacaagaagaac

SEQ ID NO: 140 - DNA, 47 bp, Artificial Sequence
  Description of Artificial Sequence DNA encoding an shRNA directed against the USP9Y gene
cttgagcttgagtgaccacttcaagagagtggtcactcaagctcaag

SEQ ID NO: 141 - DNA, 47 bp, Artificial Sequence
  Description of Artificial Sequence DNA encoding an shRNA direcetd against the gene for the ubiquitin carboxyl-terminal hydrolase 5
gaccggccagcgagtctacttcaagagagtagactcgctggccggtc

SEQ ID NO: 142 - DNA, 47 bp, Artificial Sequence
  Description of Artificial Sequence DNA encoding an shRNA directed against the gene for the ubiquitin carboxyl-terminal hydrolase 5
ggacctgggctacatctacttcaagagagtagatgtagcccaggtcc

SEQ ID NO: 143 - DNA, 47 bp, Artificial Sequence
  Description of Artificial Sequence DNA encoding an shRNA directed against the gene for the ubiquitin carboxyl-terminal hydrolase 5
ctctgtggtccaggtgctcttcaagagagagcacctggaccacagag

SEQ ID NO: 144 - DNA, 47 bp, Artificial Sequence
  Description of Artificial Sequence DNA encoding an shRNA directed against the gene for the ubiquitin carboxyl-terminal hydrolase 5
gaccacacgatttgcctcattcaagagatgaggcaaatcgtgtggtc

SEQ ID NO: 145 - DNA, 47 bp, Artificial Sequence
  Description of Artificial Sequence DNA encoding an shRNA directed against the gene for the ubiquitin carboxyl-terminal hydrolase 26
tggcttgtttattgaaggattcaagagatccttcaataaacaagcca

SEQ ID NO: 146 - DNA, 47 bp, Artificial Sequence
  Description of Artificial Sequence DNA encoding an shRNA directed against the gene for the ubiquitin carboxyl-terminal hydrolase 26
gtgaatttggggaagataattcaagagattatcttccccaaattcac

SEQ ID NO: 147 - DNA, 47 bp, Artificial Sequence
  Description of Artificial Sequence DNA encoding an shRNA directed against the gene for the ubiquitin carboxyl- terminal hydrolase 26
cgctatagcttgaatgagtttcaagagaactcattcaagctatagcg

SEQ ID NO: 148 - DNA, 47 bp, Artificial Sequence
  Description of Artificial Sequence DNA encoding an shRNA directed against the gene for the ubiquitin carboxyl- terminal hydrolase 26
gatatcctggctccacacattcaagagatgtgtggagccaggatatc

SEQ ID NO: 149 - DNA, 47 bp, Artificial Sequence
  Description of Artificial Sequence DNA encoding an shRNA directed against the KIAA1097 gene
gagccagtcggatgtagatttcaagagaatctacatccgactggctc

SEQ ID NO: 150 - DNA, 47 bp, Artificial Sequence
  Description of Artificial Sequence DNA encoding an shRNA directed against the KIAA1097 gene
gtaaattctgaaggcgaatttcaagagaattcgccttcagaatttac

SEQ ID NO: 151 - DNA, 47 bp, Artificial Sequence
  Description of Artificial Sequence DNA encoding an shRNA directed against the KIAA1097 gene
gccctcctaaatcaggcaattcaagagattgcctgatttaggagggc

SEQ ID NO: 152 - DNA, 47 bp, Artificial Sequence
  Description of Artificial Sequence DNA encoding an shRNA directed against the KIAA1097 gene
gttgagaaatggagtgaagttcaagagacttcactccatttctcaac

SEQ ID NO: 153 - DNA, 47 bp, Artificial Sequence
  Description of Artificial Sequence DNA encoding an shRNA directed against the gene for the ubiquitin specific protease (USP22) gene
gcttggaaaatgcaaggcgttcaagagacgccttgcattttccaagc

SEQ ID NO: 154 - DNA, 47 bp, Artificial Sequence
  Description of Artificial Sequence DNA encoding an shRNA directed against the gene for the ubiquitin specific protease (USP22) gene
ctgcatcatagaccagatcttcaagagagatctggtctatgatgcag

SEQ ID NO: 155 - DNA, 47 bp, Artificial Sequence
  Description of Artificial Sequence DNA encoding an shRNA directed against the gene for the ubiquitin specific protease (USP22) gene
gatcaccacgtatgtgtccttcaagagaggacacatacgtggtgatc

SEQ ID NO: 156 - DNA, 47 bp, Artificial Sequence
  Description of Artificial Sequence DNA encoding an shRNA directed against the gene for the ubiquitin specific protease 22 (USP22) gene
tgacaacaagtattccctgttcaagagacagggaatacttgttgtca

SEQ ID NO: 157 - DNA, 47 bp, Artificial Sequence
  Description of Artificial Sequence DNA encoding an shRNA directed against the gene for the ubiquitin specific processing protease 29
gaaatataagacagattccttcaagagaggaatctgtcttatatttc

SEQ ID NO: 158 - DNA, 47 bp, Artificial Sequence
  Description of Artificial Sequence DNA encoding an shRNA directed against the gene for the ubiquitin specific processing protease 29
cccatcaagtttagaggatttcaagagaatcctctaaacttgatggg

SEQ ID NO: 159 - DNA, 47 bp, Artificial Sequence
  Description of Artificial Sequence DNA encoding an shRNA directed against the gene for the ubiquitin specific processing protease 29
ggtgtcccatgggaatatattcaagagatatattcccatgggacacc

SEQ ID NO: 160 - DNA, 47 bp, Artificial Sequence
  Description of Artificial Sequence DNA encoding an shRNA directed against the gene for the ubiquitin specific processing protease 29
gaatgccgacctacaaagattcaagagatctttgtaggtcggcattc

SEQ ID NO: 161 - DNA, 47 bp, Artificial Sequence
  Description of Artificial Sequence DNA encoding an shRNA directed against the CYLD gene
cagttatattctgtgatgtttcaagagaacatcacagaatataactg

SEQ ID NO: 162 - DNA, 47 bp, Artificial Sequence
  Description of Artificial Sequence DNA encoding an shRNA directed against the CYLD gene
gaggtgttggggacaaaggttcaagagacctttgtccccaacacctc

SEQ ID NO: 163 - DNA, 47 bp, Artificial Sequence
  Description of Artificial Sequence DNA encoding an shRNA directed against the CYLD gene
gtgggctcattggctgaagttcaagagacttcagccaatgagcccac

SEQ ID NO: 164 - DNA, 47 bp, Artificial Sequence
  Description of Artificial Sequence DNA encoding an shRNA directed against the CYLD gene
gagctactgaggacagaaattcaagagatttctgtcctcagtagctc

SEQ ID NO: 165 - DNA, 47 bp, Artificial Sequence
  Description of Artificial Sequence DNA encoding an shRNA directed against the gene for the ubiquitin carboxyl-terminal hydrolase 2
tcagcaggatgctcaggagttcaagagactcctgagcatcctgctga

SEQ ID NO: 166 - DNA, 47 bp, Artificial Sequence
  Description of Artificial Sequence DNA encoding an shRNA directed against the gene for  the ubiquitin carboxyl-terminal hydrolase 2
gaagttctccatccagaggttcaagagacctctggatggagaacttc

SEQ ID NO: 167 - DNA, 47 bp, Artificial Sequence
  Description of Artificial Sequence DNA encoding an shRNA directed against the gene for the ubiquitin carboxyl-terminal hydrolase 2
gccggtccccaccagcagcttcaagagagctgctggtggggaccggc

SEQ ID NO: 168 - DNA, 47 bp, Artificial Sequence
  Description of Artificial Sequence DNA encoding an shRNA directed against the gene for the ubiquitin carboxyl-terminal hydrolase 2
cactcgggagttgagagatttcaagagaatctctcaactcccgagtg

SEQ ID NO: 169 - DNA, 47 bp, Artificial Sequence
  Description of Artificial Sequence DNA encoding an shRNA directed against the gene for the ubiquitin specific protease 3 (USP3)
gcccttgggtctgtttgacttcaagagagtcaaacagacccaagggc

SEQ ID NO: 170 - DNA, 47 bp, Artificial Sequence
  Description of Artificial Sequence DNA encoding an shRNA directed against the gene for the ubiquitin specific protease 3 (USP3)
ctcaacactaaacagcaagttcaagagacttgctgtttagtgttgag

SEQ ID NO: 171 - DNA, 47 bp, Artificial Sequence
  Description of Artificial Sequence DNA encoding an shRNA directed against the gene for the ubiquitin specific protease 3 (USP3)
gatttcattggacagcatattcaagagatatgctgtccaatgaaatc

SEQ ID NO: 172 - DNA, 47 bp, Artificial Sequence
  Description of Artificial Sequence DNA encoding an shRNA directed against the gene for the ubiquitin specific protease 3 (USP3)
catggggcaccaactaattttcaagagaaattagttggtgccccatg

SEQ ID NO: 173 - DNA, 47 bp, Artificial Sequence
  Description of Artificial Sequence DNA encoding an shRNA directed against the gene for the ubiquitin carboxyl-terminal hydrolase 23
ggtgtctctgcgggattgtttcaagagaacaatcccgcagagacacc

SEQ ID NO: 174 - DNA, 47 bp, Artificial Sequence
  Description of Artificial Sequence DNA encoding an shRNA directed against the gene for the ubiquitin carboxyl-terminal hydrolase 23
agttcagtaggtgtagactttcaagagaagtctacacctactgaact

SEQ ID NO: 175 - DNA, 47 bp, Artificial Sequence
  Description of Artificial Sequence DNA encoding an shRNA directed against the gene for the ubiquitin carboxyl-terminal hydrolase 23
gagttcctgaagctcctcattcaagagatgaggagcttcaggaactc

SEQ ID NO: 176 - DNA, 47 bp, Artificial Sequence
  Description of Artificial Sequence DNA encoding an shRNA directed against the gene for the ubiquitin carboxyl-terminal hydrolase 23
ggatttgctgggggcaaggttcaagagaccttgcccccagcaaatcc

SEQ ID NO: 177 - DNA, 47 bp, Artificial Sequence
  Description of Artificial Sequence DNA encoding an shRNA directed against the UBP-32.7 gene
ctcagaaagccaacattcattcaagagatgaatgttggctttctgag

SEQ ID NO: 178 - DNA, 47 bp, Artificial Sequence
  Description of Artificial Sequence DNA encoding an shRNA directed against the UBP-32.7 gene
cgcattgtaataagaaggtttcaagagaaccttcttattacaatgcg

SEQ ID NO: 179 - DNA, 47 bp, Artificial Sequence
  Description of Artificial Sequence DNA encoding an shRNA directed against the UBP-32.7 gene
gggaggaaaatgcagaaatttcaagagaatttctgcattttcctccc

SEQ ID NO: 180 - DNA, 47 bp, Artificial Sequence
  Description of Artificial Sequence DNA encoding an shRNA directed against the UBP-32.7 gene
ttacaaatttaggaaatacttcaagagagtatttcctaaatttgtaa

SEQ ID NO: 181 - DNA, 47 bp, Artificial Sequence
  Description of Artificial Sequence DNA encoding an shRNA directed against the gene for the Homo sapiens ubiquitin specific protease 13 (isopeptidase T-3)
gttatgaattgatatgcagttcaagagactgcatatcaattcataac

SEQ ID NO: 182 - DNA, 47 bp, Artificial Sequence
  Description of Artificial Sequence DNA encoding an shRNA directed against the gene for the Homo sapiens ubiquitin specific protease 13 (isopeptidase T-3)
gtgataacacaactaatggttcaagagaccattagttgtgttatcac

SEQ ID NO: 183 - DNA, 47 bp, Artificial Sequence
  Description of Artificial Sequence DNA encoding an shRNA directed against the gene for the Homo sapiens ubiquitin specific protease 13 (isopeptidase T-3)
gtagaggagagttctgaaattcaagagatttcagaactctcctctac

SEQ ID NO: 184 - DNA, 47 bp, Artificial Sequence
  Description of Artificial Sequence DNA encoding an shRNA directed against the gene for the Homo sapiens ubiquitin specific protease 13 (isopeptidase T-3)
gcctctaatcctgataaggttcaagagaccttatcaggattagaggc

SEQ ID NO: 185 - DNA, 47 bp, Artificial Sequence
  Description of Artificial Sequence DNA encoding an shRNA directed against the gene for the ubiquitin carboxyl-terminal hydrolase 28
gatgatcttcaggctgccattcaagagatggcagcctgaagatcatc

SEQ ID NO: 186 - DNA, 47 bp, Artificial Sequence
  Description of Artificial Sequence DNA encoding an shRNA directed against the gene for the ubiquitin carboxyl-terminal hydrolase 28
gtatggacaagagcgttggttcaagagaccaacgctcttgtccatac

SEQ ID NO: 187 - DNA, 47 bp, Artificial Sequence
  Description of Artificial Sequence DNA encoding an shRNA directed against the gene for the ubiquitin carboxyl-terminal hydrolase 28
cgaacccttctggaacagtttcaagagaactgttccagaagggttcg

SEQ ID NO: 188 - DNA, 47 bp, Artificial Sequence
  Description of Artificial Sequence DNA encoding an shRNA directed against the gene for the ubiquitin carboxyl-terminal hydrolase 28
gtggcatgaagattatagtttcaagagaactataatcttcatgccac

SEQ ID NO: 189 - DNA, 47 bp, Artificial Sequence
  Description of Artificial Sequence DNA encoding an shRNA directed against the ubiquitin carboxyl-terminal hydrolase 14
ggtgaacaaggacagtatcttcaagagagatactgtccttgttcacc

SEQ ID NO: 190 - DNA, 47 bp, Artificial Sequence
  Description of Artificial Sequence DNA encoding an shRNA directed against the gene for the ubiquitin carboxyl-terminal hydrolase 14
gcaatagaggatgattctgttcaagagacagaatcatcctctattgc

SEQ ID NO: 191 - DNA, 47 bp, Artificial Sequence
  Description of Artificial Sequence DNA encoding an shRNA directed against the gene for the ubiquitin carboxyl-terminal hydrolase 14
tctgtgaatgccaaagttcttcaagagagaactttggcattcacaga

SEQ ID NO: 192 - DNA, 47 bp, Artificial Sequence
  Description of Artificial Sequence DNA encoding an shRNA directed against the gene for the ubiquitin carboxyl-terminal hydrolase 14
cacaccagggaaggtctagttcaagagactagaccttccctggtgtg

SEQ ID NO: 193 - DNA, 47 bp, Artificial Sequence
  Description of Artificial Sequence DNA encoding an shRNA directed against the DUB1 gene
gcaggaagatgcccatgaattcaagagattcatgggcatcttcctgc

SEQ ID NO: 194 - DNA, 47 bp, Artificial Sequence
  Description of Artificial Sequence DNA encoding an shRNA directed agains the DUB1 gene
gaatgtgcaatatcctgagttcaagagactcaggatattgcacattc

SEQ ID NO: 195 - DNA, 47 bp, Artificial Sequence
  Description of Artificial Sequence DNA encoding an shRNA directed against the DUB1 gene
tggatgatgccaaggtcacttcaagagagtgaccttggcatcatcca

SEQ ID NO: 196 - DNA, 47 bp, Artificial Sequence
  Description of Artificial Sequence DNA encoding an shRNA directed against the DUB1 gene
gctccgtgctaaacctctcttcaagagagagaggtttagcacggagc

SEQ ID NO: 197 - DNA, 47 bp, Artificial Sequence
  Description of Artificial Sequence DNA encoding an shRNA directed against the gene for the mouse USP27 homolog
gcctccacctcaacagaggttcaagagacctctgttgaggtggaggc

SEQ ID NO: 198 - DNA, 47 bp, Artificial Sequence
  Description of Artificial Sequence DNA encoding an shRNA directed against the gene for the mouse USP27 homolog
ctgcatcatagaccaaatcttcaagagagatttggtctatgatgcag

SEQ ID NO: 199 - DNA, 47 bp, Artificial Sequence
  Description of Artificial Sequence DNA encoding an shRNA directed against the gene for the mouse USP27 homolog
gatcactacatacatttccttcaagagaggaaatgtatgtagtgatc

SEQ ID NO: 200 - DNA, 47 bp, Artificial Sequence
  Description of Artificial Sequence DNA encoding an shRNA directed against the gene for the mouse USP27 homolog
gtaaagagagcagaatgaattcaagagattcattctgctctctttac

SEQ ID NO: 201 - DNA, 47 bp, Artificial Sequence
  Description of Artificial Sequence DNA encoding an shRNA directed against the gene for the ubiquitin carboxyl-terminal hydrolase 4
cgcggggcgcagtggtatcttcaagagagataccactgcgccccgcg

SEQ ID NO: 202 - DNA, 47 bp, Artificial Sequence
  Description of Artificial Sequence DNA encoding an shRNA directed against the gene for the ubiquitin carboxyl-terminal hydrolase 4
cagaaggcagtggggaagattcaagagatcttccccactgccttctg

SEQ ID NO: 203 - DNA, 47 bp, Artificial Sequence
  Description of Artificial Sequence DNA encoding an shRNA directed against the gene for the ubiquitin carboxyl-terminal hydrolase 4
gcctgggagaatcacaggtttcaagagaacctgtgattctcccaggc

SEQ ID NO: 204 - DNA, 47 bp, Artificial Sequence
  Description of Artificial Sequence DNA encoding an shRNA directed against the gene for the ubiquitin carboxyl-terminal hydrolase 4
accagacaaggaaatacccttcaagagagggtatttccttgtctggt

SEQ ID NO: 205 - DNA, 47 bp, Artificial Sequence
  Description of Artificial Sequence DNA encoding an shRNA directed against the TRE-2 gene
cacatccaccacatcgaccttcaagagaggtcgatgtggtggatgtg

SEQ ID NO: 206 - DNA, 47 bp, Artificial Sequence
  Description of Artificial Sequence DNA encoding an shRNA directed against the TRE-2 gene
gtcacaacccaagaccatgttcaagagacatggtcttgggttgtgac

SEQ ID NO: 207 - DNA, 47 bp, Artificial Sequence
  Description of Artificial Sequence DNA encoding an shRNA directed against the TRE-2 gene
ctcaacaggacaaatcccattcaagagatgggatttgtcctgttgag

SEQ ID NO: 208 - DNA, 47 bp, Artificial Sequence
  Description of Artificial Sequence DNA encoding an shRNA directed against the TRE-2 gene
tagatcaattattgtggatttcaagagaatccacaataattgatcta

SEQ ID NO: 209 - DNA, 47 bp, Artificial Sequence
  Description of Artificial Sequence DNA encoding an shRNA directed against the gene for the ubiquitin carboxyl-terminal hydrolase 15 (UNPH-2)
ggaacaccttattgatgaattcaagagattcatcaataaggtgttcc

SEQ ID NO: 210 - DNA, 47 bp, Artificial Sequence
  Description of Artificial Sequence DNA encoding an shRNA directed against the gene for the ubiquitin carboxyl-terminal hydrolase 15 (UNPH-2)
ctttaacagaaattgtctcttcaagagagagacaatttctgttaaag

SEQ ID NO: 211 - DNA, 47 bp, Artificial Sequence
  Description of Artificial Sequence DNA encoding an shRNA directed agaainst the gene for the ubiquitin carboxyl-terminal hydrolase 15 (UNPH-2)
cctatgcagtacaaagtggttcaagagaccactttgtactgcatagg

SEQ ID NO: 212 - DNA, 47 bp, Artificial Sequence
  Description of Artificial Sequence DNA encoding an shRNA directed against the gene for the ubiquitin carboxyl-terminal hydrolase 15 (UNPH-2)
gatcttttcttgctttggattcaagagatccaaagcaagaaaagatc

SEQ ID NO: 213 - DNA, 47 bp, Artificial Sequence
  Description of Artificial Sequence DNA encoding an shRNA directed against the KIAA1372 gene
cagcatccttcaggccttattcaagagataaggcctgaaggatgctg

SEQ ID NO: 214 - DNA, 47 bp, Artificial Sequence
  Description of Artificial Sequence DNA encoding an shRNA directed against the KIAA1372 gene
gatagtgactcggatctgcttcaagagagcagatccgagtcactatc

SEQ ID NO: 215 - DNA, 47 bp, Artificial Sequence
  Description of Artificial Sequence DNA encoding an shRNA directed against the KIAA1372 gene
gacatcacagcccgggagtttcaagagaactcccgggctgtgatgtc

SEQ ID NO: 216 - DNA, 47 bp, Artificial Sequence
  Description of Artificial Sequence DNA encoding an shRNA directed against the KIAA1372 gene
ggacacagcctatgtgctgttcaagagacagcacataggctgtgtcc

SEQ ID NO: 217 - DNA, 47 bp, Artificial Sequence
  Description of Artificial Sequence DNA encoding an shRNA directed against the gene for the BRCA1 associated protein-1
gtggaggagatctacgaccttcaagagaggtcgtagatctcctccac

SEQ ID NO: 218 - DNA, 47 bp, Artificial Sequence
  Description of Artificial Sequence DNA encoding an shRNA directed against the gene for the BRCA1 associated protein-1
ctcttgtgcaactcatgccttcaagagaggcatgagttgcacaagag

SEQ ID NO: 219 - DNA, 47 bp, Artificial Sequence
  Description of Artificial Sequence DNA encoding an shRNA directed against the gene for the BRCA1 associated protein-1
acagggcccctgcagcctcttcaagagagaggctgcaggggccctgt

SEQ ID NO: 220 - DNA, 47 bp, Artificial Sequence
  Description of Artificial Sequence DNA encoding an shRNA directed against the gene for the BRCA1 associated protein-1
gaagacctggcggcaggtgttcaagagacacctgccgccaggtcttc

SEQ ID NO: 221 - DNA, 4167 bp, Mus musculus
gccggcgtggcgtgctctgatcgccggggtcccagcactcctactgctatgggcttcgggagaggatgtgagacgacggctgtgccattgctggtggccgtggccgcgttgctggtgggcacagccggccacctgtaccctggagaggtgtgccctggtatggacatccggaacaacctgaccaggctacatgagctggagaactgctcagtcattgagggccatctgcagatcctcctgatgttcaagaccagacccgaagatttccgagacctcagtttccccaaactcatcatgatcacagattacctgcttctcttccgtgtctatggtctggaaagtctgaaagacctcttcccaaatctcacagtcatccgaggctcccgtctcttcttcaactatgccctggttatcttcgagatggtccacctgaaggagctggggctttataacctcatgaacatcacccggggctctgtccgcatcgagaagaataatgagctctgctacctggccactatcgactggtcccgtatcctggattctgtggaggacaactacattgtactgaacaaagatgacaacgaggaatgtggggatgtctgtccaggcaccgccaagggcaagaccaactgtcctgccactgtcatcaatgggcagtttgtggaacggtgctggacacacagtcattgtcagaaagtttgcccaaccatctgtaagtcacatggctgcacagctgaaggcctgtgctgccacaaagagtgcctgggcaactgttcggaacctgatgaccccaccaagtgtgtggcctgtcgcaacttctatctggatggtcagtgtgtggagacctgcccgccaccctactatcacttccaggactggcgctgtgtgaacttcagcttctgccaagaccttcacttcaaatgcaggaactctcggaagcctggctgccaccaatacgtcattcacaacaataagtgcatccccgagtgcccgtctggctataccatgaattccagcaacttgatgtgcaccccatgtctgggaccctgccctaaggtctgccaaatcctcgaaggtgagaagaccattgattctgtgacatctgcccaggagctccgaggctgcactgtgatcaacggtagcctgatcatcaacatccgagggggcaacaacctggcagctgagctggaggctaaccttggcctcattgaagaaatttcgggatttctaaagatccgccgctcctatgctctggtatcactttctttcttcaggaagctacatctgattcgaggagagaccttggaaattgggaactattctttttatgccttggacaaccagaacctgaggcaactctgggactggagcaaacacaacctcaccatcactcagggcaagctcttcttccattacaatccgaaactctgcttgtctgaaattcacaagatggaagaagtctccggaactaagggccgtcaggagaggaacgacattgccctgaagaccaatggggaccaggcatcgtgtgaaaatgaattgcttaaattttctttcattcggacatcttttgacaagatcctgttgaggtgggaaccctactggccccccgacttccgagatctcctgggattcatgttgttctacaaagaggccccttatcagaatgtgacagagtttgatgggcaggatgcttgtggctccaacagctggactgtggtggatattgacccgccccagaggtccaacgaccccaagtctcagaccccaagccaccctgggtggctgatgcggggcctcaaaccctggacccaatacgccatctttgtgaagaccttggttaccttctctgatgaacggcggacctatggagccaaaagtgatatcatctatgtgcaaacagatgccactaatccttctgtccccctggatcccatatcagtttctaattcctcatctcagattatcttaaagtggaagcccccctctgaccccaatggcaacatcacacactacctggtgtactgggagaggcaagcagaggacagcgagctgtttgagctggattattgtctcaaagggctgaagctcccttcacggacctggtccccaccctttgagtctgatgattctcagaagcacaatcagagtgagtatgacgactcggccagtgagtgctgctcatgccctaagactgactctcagatcctgaaggagctggaggagtcttcattcaggaagaccttcgaggattacctgcacaacgtggtttttgtccccaggccatcccgaaagcgaagatcccttgaagaggtggggaatgtgacagccaccacactcacacttccagatttccccaacgtctcctctaccattgtgcccacaagtcaggaggagcacaggccatttgagaaagtggtgaacaaggagtcacttgtcatctctggcctgagacacttcactgggtaccgcattgagctgcaggcatgcaatcaagattccccagatgagaggtgcagtgtggctgcctacgtcagtgcccggaccatgcctgaagctaaggcagatgacatcgttggccctgtgactcatgaaatctttgagaacaatgttgtacacttaatgtggcaagagccaaaggaacctaatggtctgattgtgctatatgaagtgagctatcgccgatatggtgatgaggagctgcacctctgtgtctcccggaagcattttgccctggagcggggctgcaggctgcgagggctctccccaggaaactacagtgttcgagtccgggctacctctctggcaggaaatggctcctggacagaacccacctatttttatgtgactgattatttagatgtcccatcaaatattgccaaaattatcattggacccctcatctttgtcttcctcttcagtgttgtgattggaagtatttatctatttctgagaaagaggcagccggatgggccaatgggaccactgtatgcatcttcaaaccctgagtacctcagtgccagtgatgtgtttccatcttctgtgtacgtgccggacgagtgggaggtgcctcgagagaagatcacccttcttcgagagctggggcagggatcctttggtatggtgtatgaaggcaatgccaaggatatcatcaagggtgaggcagagacccgtgttgcggttaagactgtcaatgagtcagccagtcttcgagaacggatcgagttcctcaatgaggcatcagtcatgaagggattcacctgccatcatgtggtccgccttcttggggtggtatccaaaggacagccaatgctggtagtgatggaattgatggctcatggagacctgaaaagtcacctccgttctctgaggccagatgctgagaataacccaggccgccctccccctaccttgcaagaaatgattcagatgacagcagaaattgctgatggcatggcatacttgaacgccaagaagtttgtgcaccgggacctggcagctcgaaactgcatggttgcccatgattttactgtcaaaattggagactttggaatgacaagggacatctacgagacagattactatcggaaagggggcaagggactgcttcctgtgaggtggatgtcacctgagtccctgaaggatggagtctttactgcttcttctgatatgtggtcctttggggtggtcctttgggaaatcactagcttggctgagcaaccttatcaaggcctgtctaatgaacaggtgttgaagtttgtcatggatggaggctatctggatccccctgataactgtccagagagactcactgacctgatgcgcatgtgctggcagttcaaccccaagatgaggccaaccttcctggaaatcgtcaacctgctcaaggatgacctccaccccagctttccagaagtttccttcttctacagcgaggagaacaaggctcctgagagtgaggagctggagatggagtttgaagacatggagaatgtcccgttggatcgttcctctcactgtcagagagaagaggctgggggccgggagggagggtcctcactgagcatcaaacggacctatgatgaacacatcccctatacccacatgaatgggggcaagaagaacggacgtgtccttaccctgccaaggtcaaacccttcctaa

SEQ ID NO: 222 - DNA, 3327 bp, Artificial Sequence
  Description of Artificial Sequence vector pIR5
ccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagtccctatcagtgatagagattcccagaccagacccgaagatttcttcaagagagaaatcttcgggtctggtcttttttggcgcgccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattggagctcgatatcggccgg

SEQ ID NO: 223 - DNA, 8507 bp, Artificial Sequence
  Description of Artificial Sequence vector pIR5-tet
atccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccggagaagttcctattccgaagttcctattcttcaaatagtataggaacttcgctcgagggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatggcgcgtatgcattaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatcctcacagtaggtggcatcgttcctttctgactgcccgccccccgcatgccgtcccgcgatattgagctccgaacctctcgccctgccgccgccggtgctccgtcgccgccgcgccgccatggaattcgaacgctgacgtcatcaacccgctccaaggaatcgcgggcccagtgtcactaggcgggaacacccagcgcgcgtgcgccctggcaggaagatggctgtgagggacaggggagtggcgccctgcaatatttgcatgtcgctatgtgttctgggaaatcaccataaacgtgaaatgtctttggatttgggaatcttataagtccctatcagtgatagagattcccagaccagacccgaagatttcttcaagagagaaatcttcgggtctggtcttttttggttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcggctctagaagcgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacattgacatccactttgcctttctctccacaggtgtccactcccagggcggcctccggagcgatcgccggtccgcctaggcaattcaccatgtccagactggacaagagcaaagtcatcaactctgccttggagctcctgaatgaagttggcattgagggcttgaccaccaggaagctggcccagaagctgggtgtggagcagcctaccctgtactggcatgtgaagaacaagagggctctgcttgatgccctggccattgagatgttggacaggcaccacacccacttctgccctctggaaggggagtcctggcaggacttcctgaggaacaatgccaagagcttcagatgtgccttgctctcccaccgggatggtgccaaagttcacttgggcaccaggcctacagagaagcagtatgagaccctggagaaccagctggcattcctgtgccaacaaggcttctccctggaaaatgccttgtatgccctctctgctgtgggccacttcaccttgggctgtgtgctggaggaccaggagcaccaagttgccaaggaggagagggagacccccaccactgactccatgccaccactgctgcggcaagctattgagttgtttgaccaccaaggggctgagcctgcattcctttttggcctggaactgatcatctgtggcctggaaaagcagctgaaatgtgagtctggctcttaaggatccatcgattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcgccggtaaccgaagttcctatactttctagagaataggaacttcggaataggaacttcaagccggtacccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggggtaactaagtaaggatcgag

SEQ ID NO: 224 - DNA, 49 bp, Artificial Sequence
  Description of Artificial Sequence shRNA sequence
agtccgcatcgagaagaatattcaagagatattcttctcgatgcggact

SEQ ID NO: 225 - DNA, 49 bp, Artificial Sequence
  Description of Artificial Sequence shRNA sequence
atcgagaagaataatgagctttcaagagaagctcattattcttctcgat

SEQ ID NO: 226 - DNA, 47 bp, Artificial Sequence
  Description of Artificial Sequence shRNA sequence
actacattgtactgaacaattcaagagattgttcagtacaatgtagt

SEQ ID NO: 227 - DNA, 49 bp, Artificial Sequence
  Description of Artificial Sequence shRNA sequence
agggcaagaccaactgtcctttcaagagaaggacagttggtcttgccct

SEQ ID NO: 228 - DNA, 49 bp, Artificial Sequence
  Description of Artificial Sequence shRNA sequence
agaccagacccgaagatttcttcaagagagaaatcttcgggtctggtct

SEQ ID NO: 229 - DNA, 49 bp, Artificial Sequence
  Description of Artificial Sequence shRNA sequence
agcctggctgccaccaatacttcaagagagtattggtggcagccaggct

SEQ ID NO: 230 - DNA, 28 bp, Artificial Sequence
  Description of Artificial Sequence primer
atcgggatccagtggaaagacgcgcagg

SEQ ID NO: 231 - DNA, 59 bp, Artificial Sequence
  Description of Artificial Sequence primer
gctctagaagaccactttctctatcactgatagggagatatataaagccaagaaatcga

SEQ ID NO: 232 - DNA, 264 bp, Artificial Sequence
  Description of Artificial Sequence U6-tet promoter
aaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatctccctatcagtgatagagaaa

SEQ ID NO: 233 - DNA, 69 bp, Artificial Sequence
  Description of Artificial Sequence Fluc- specific shRNA
gggattccaattcagcgggagccacctgatgaagcttgatcgggtggctctcgctgagttggaatccat

SEQ ID NO: 234 - DNA, 15174 bp, Artificial Sequence
  Description of Artificial Sequence U6-tet targeting vector
ctagggataacagggtaatatagccgcggcaggccctccgagcgtggtggagccgttctgtgagacagccgggtacgagtcgtgacgctggaaggggcaagcgggtggtgggcaggaatgcggtccgccctgcagcaaccggagggggagggagaagggagcggaaaagtctccaccggacgcggccatggctcgggggggggggggcagcggaggagcgcttccggccgacgtctcgtcgctgattggcttcttttcctcccgccgtgtgtgaaaacacaaatggcgtgttttggttggcgtaaggcgcctgtcagttaacggcagccggagtgcgcagccgccggcagcctcgctctgcccactgggtggggcgggaggtaggtggggtgaggcgagctggacgtgcgggcgcggtcggcctctggcggggcgggggaggggagggagggtcagcgaaagtagctcgcgcgcgagcggccgcccaccctccccttcctctgggggagtcgttttacccgccgccggccgggcctcgtcgtctgattggctctcggggcccagaaaactggcccttgccattggctcgtgttcgtgcaagttgagtccatccgccggccagcgggggcggcgaggaggcgctcccaggttccggccctcccctcggccccgcgccgcagagtctggccgcgcgcccctgcgcaacgtggcaggaagcgcgcgctgggggcggggacgggcagtagggctgagcggctgcggggcgggtgcaagcacgtttccgacttgagttgcctcaagaggggcgtgctgagccagacctccatcgcgcactccggggagtggagggaaggagcgagggctcagttgggctgttttggaggcaggaagcacttgctctcccaaagtcgctctgagttgttatcagtaagggagctgcagtggagtaggcggggagaaggccgcacccttctccggaggggggaggggagtgttgcaatacctttctgggagttctctgctgcctcctggcttctgaggaccgccctgggcctgggagaatcccttccccctcttccctcgtgatctgcaactccagtctttctaggtaaccgatatccctgcaggggtgacctgcacgtctagggcgcagtagtccagggtttccttgatgatgtcatacttatcctgtcccttttttttccacagctcgcggttgaggacaaactcttcgcggtctttccagtactcctgcaggtgactgactgagtcgagatctgcgatctaagtaagcttggcattccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagaccggttcgagatccaggcgcggatcaataaaagatcattattttcaatagatctgtgtgttggttttttgtgtgccttgggggagggggaggccagaatgaggcgcggccaagggggagggggaggccagaatgaccttgggggagggggaggccagaatgaccttgggggagggggaggccagaatgaggcgcggatccgtcgacttaattaaggccagggatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgtagggcctgcggggtctattgggaaccaagctggagtgcagtggcacaatcttggctcactgcaatctccgcctcctgggttcaagcgattctcctgcctcagcctcccgagttgttgggattccaggcatgcatgaccaggctcagctaatttttgtttttttggtagagacggggtttcaccatattggccaggctggtctccaactcctaatctcaggtgatctacccaccttggcctcccaaattgctgggattacaggcgtgaaccactgctcccttccctgtccttctgattttaaaataactataccagcaggaggacgtccagacacagcataggctacctggccatgcccaaccggtgggacatttgagttgcttgcttggcactgtcctctcatgcgttgggtccactcagtagatgcctgttgaattaagcttatttaaataggccggccataacttcgtataatgtatgctatacgaagttatggatccagtggaaagacgcgcaggcaaaacgcaccacgtgacggagcgtgaccgcgcgccgagcccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatctccctatcagtgatagagaaagggattccaattcagcgggagccacctgatgaagcttgatcgggtggctctcgctgagttggaatccatttttttctagactcgagataacttcgtataatgtatgctatacgaagttatggcgcgccggtaaccgaagttcctatactttctagagaataggaacttcggaataggaacttcttaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcgatccgctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctagatcccgcgccgaagttcctatactttctagagaataggaacttcggaataggaacttcaagcttaagcgctagaagatgggcgggagtcttctgggcaggcttaaaggctaacctggtgtgtgggcgttgtcctgcaggggaattgaacaggtgtaaaattggagggacaagacttcccacagattttcggttttgtcgggaagttttttaataggggcaaataaggaaaatgggaggataggtagtcatctggggttttatgcagcaaaactacaggttattattgcttgtgatccgcctcggagtattttccatcgaggtagattaaagacatgctcacccgagttttatactctcctgcttgagatccttactacagtatgaaattacagtgtcgcgagttagactatgtaagcagaattttaatcatttttaaagagcccagtacttcatatccatttctcccgctccttctgcagccttatcaaaaggtattttagaacactcattttagccccattttcatttattatactggcttatccaacccctagacagagcattggcattttccctttcctgatcttagaagtctgatgactcatgaaaccagacagattagttacatacaccacaaatcgaggctgtagctggggcctcaacactgcagttcttttataactccttagtacactttttgttgatcctttgccttgatccttaattttcagtgtctatcacctctcccgtcagtggtgttccacatttgggcctattctcagtccagggagttttacaacaatagatgtattgagaatccaacctaaagcttaactttccactcccatgaatgcctctctcctttttctccatttataaactgagctattaaccattaatggttccaggtggatgtctcctccccatattacctgatgtatcttacatattgccaggctgatattttaagacattaaaaggtatatttcattattgagccacatggtattgattactgcttactaaaattttgtcattgtacacatctgtaaaaggtggttccttttggaatgcaaagttcaggtgtttgttgtctttcctgacctaaggtcttgtgagcttgtattttttctatttaagcagtgctttctcttggactggcttgactcatggcattctacacgttattgctggtctaaatgtgattttgccaagcttcttcaggacctataattttgcttgacttgtagccaaacacaagtaaaatgattaagcaacaaatgtatttgtgaagcttggtttttaggttgttgtgttgtgtgtgcttgtgctctataataatactatccaggggctggagaggtggctcggagttcaagagcacagactgctcttccagaagtcctgagttcaattcccagcaaccacatggtggctcacaaccatctgtaatgggatctgatgccctcttctggtgtgtctgaagaccacaagtgtattcacattaaataaataaatcctccttcttcttcttttttttttttttaaagagaatactgtctccagtagaatttactgaagtaatgaaatactttgtgtttgttccaatatggtagccaataatcaaattactctttaagcactggaaatgttaccaaggaactaatttttatttgaagtgtaactgtggacagaggagccataactgcagacttgtgggatacagaagaccaatgcagactttaatgtcttttctcttacactaagcaataaagaaataaaaattgaacttctagtatcctatttgtttaaactgctagctttacttaacttttgtgcttcatctatacaaagctgaaagctaagtctgcagccattactaaacatgaaagcaagtaatgataattttggatttcaaaaatgtagggccagagtttagccagccagtggtggtgcttgcctttatgcctttaatcccagcactctggaggcagagacaggcagatctctgagtttgagcccagcctggtctacacatcaagttctatctaggatagccaggaatacacacagaaaccctgttggggaggggggctctgagatttcataaaattataattgaagcattccctaatgagccactatggatgtggctaaatccgtctacctttctgatgagatttgggtattattttttctgtctctgctgttggttgggtcttttgacactgtgggctttctttaaagcctccttcctgccatgtggtctcttgtttgctactaacttcccatggcttaaatggcatggctttttgccttctaagggcagctgctgagatttgcagcctgatttccagggtggggttgggaaatctttcaaacactaaaattgtcctttaattttttttttaaaaaatgggttatataataaacctcataaaatagttatgaggagtgaggtggactaatattaaatgagtccctcccctataaaagagctattaaggctttttgtcttatacttaactttttttttaaatgtggtatctttagaaccaagggtcttagagttttagtatacagaaactgttgcatcgcttaatcagattttctagtttcaaatccagagaatccaaattcttcacagccaaagtcaaattaagaatttctgacttttaatgttaatttgcttactgtgaatataaaaatgatagcttttcctgaggcagggtctcactatgtatctctgcctgatctgcaacaagatatgtagactaaagttctgcctgcttttgtctcctgaatactaaggttaaaatgtagtaatacttttggaacttgcaggtcagattcttttataggggacacactaagggagcttgggtgatagttggtaaaatgtgtttcaagtgatgaaaacttgaattattatcaccgcaacctactttttaaaaaaaaaagccaggcctgttagagcatgcttaagggatccctaggacttgctgagcacacaagagtagttacttggcaggctcctggtgagagcatatttcaaaaaacaaggcagacaaccaagaaactacagttaaggttacctgtctttaaaccatctgcatatacacagggatattaaaatattccaaataatatttcattcaagttttcccccatcaaattgggacatggatttctccggtgaataggcagagttggaaactaaacaaatgttggttttgtgatttgtgaaattgttttcaagtgatagttaaagcccatgagatacagaacaaagctgctatttcgaggtctcttggtttatactcagaagcacttctttgggtttccctgcactatcctgatcatgtgctaggcctaccttaggctgattgttgttcaaataaacttaagtttcctgtcaggtgatgtcatatgatttcatatatcaaggcaaaacatgttatatatgttaaacatttgtacttaatgtgaaagttaggtctttgtgggtttgatttttaattttcaaaacctgagctaaataagtcatttttacatgtcttacatttggtggaattgtataattgtggtttgcaggcaagactctctgacctagtaaccctacctatagagcactttgctgggtcacaagtctaggagtcaagcatttcaccttgaagttgagacgttttgttagtgtatactagtttatatgttggaggacatgtttatccagaagatattcaggactatttttgactgggctaaggaattgattctgattagcactgttagtgagcattgagtggcctttaggcttgaattggagtcacttgtatatctcaaataatgctggccttttttaaaaagcccttgttctttatcaccctgttttctacataatttttgttcaaagaaatacttgtttggatctccttttgacaacaatagcatgttttcaagccatattttttttcctttttttttttttttttggtttttcgagacagggtttctctgtatagccctggctgtcctggaactcactttgtagaccaggctggcctcgaactcagaaatccgcctgcctctgcctcctgagtgccgggattaaaggcgtgcaccaccacgcctggctaagttggatattttgttatataactataaccaatactaactccactgggtggatttttaattcagtcagtagtcttaagtggtctttattggcccttcattaaaatctactgttcactctaacagaggctgttggtactagtggcacttaagcaacttcctacggatatactagcagattaagggtcagggatagaaactagtctagcgttttgtatacctaccagctttatactaccttgttctgatagaaatatttcaggacatctagcacgtgttaactcgagctgcaggattcgagggccccggcaggtcaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctgcagccaatgcaccgtccttgccatcatggcctcgtaccccggccatcaacacgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgaggggatcgatccgtcctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctcccactcatgatctatagatctatagatctctcgtgggatcattgtttttctcttgattcccactttgtggttctaagtactgtggtttccaaatgtgtcagtttcatagcctgaagaacgagatcagcagcctctgttccacatacacttcattctcagtattgttttgccaagttctaattccatcagaagctgactctaggccgagctccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgtcgagatctagatatcgatggccatagagttacg

(19) United States
(12) Patent Application Publication
(10) Pub. No.: US 2009/0210955 A1
(43) Pub. Date: Aug. 20, 2009

(54) SHRNA AND SIRNA EXPRESSION IN A LIVING ORGANISM UNDER CONTROL OF A CODON-OPTIMIZED REPRESSOR GENE

(75) Inventors: Jost Seibler, Köln (DE); Frieder Schwenk, Köln (DE); Birgit Küter-Luks, Köln (DE)

Correspondence Address: NORRIS, MCLAUGHLIN & MARCUS, PA, 875 THIRD AVENUE, 18TH FLOOR, NEW YORK, NY, 10022, US

(73) Assignee: ARTEMIS PHARMACEUTICALS GMBH, KOLN (DE)

(21) Appl. No.: 11/912,451

(22) PCT Filed: Jun. 8, 2006
(86) PCT No.: PCT/EP2006/063001

§ 371 Date: Dec. 20, 2007

(30) Foreign Application Priority Data
Jun. 9, 2005 (EP) 05105076.3
Mar. 7, 2006 (EP) 06110759.5

(51) Int. Cl. A01K 67/00; C12N 5/00; C12N 5/06; C12N 15/00

(52) U.S. Cl. 800/18; 435/325; 435/354; 800/8; 435/455

(57) ABSTRACT

The present invention relates to a biological entity carrying a regulator construct comprising a specific repressor gene and a responder construct comprising at least one segment corresponding to a short hairpin RNA (shRNA) or corresponding to complementary short interfering RNA (siRNA) strands, said at least one segment being under control of a promoter which contains an operator sequence corresponding to the repressor. The invention further relates to a method for preparing said biological entity and its use.

The present invention relates to a biological entity carrying a regulator construct comprising a specific repressor gene and a responder construct comprising at least one segment corresponding to a short hairpin RNA (shRNA) or corresponding to complementary short interfering RNA (siRNA) strands, said at least one segment being under control of a promoter which contains an operator sequence corresponding to the repressor. The invention further relates to a method for preparing said biological entity and its use.

### BACKGROUND OF THE INVENTION

RNA interference (RNAi) has been discovered some years ago as a tool for inhibition of gene expression (Fire, A. et al., Nature 391, 806-811 (1998)). It based on the introduction of double stranded RNA (dsRNA) molecules into cells, whereby one strand is complementary to the coding region of a target gene. Through pairing of the specific mRNA with the introduced RNA molecule, the mRNA is degraded by a cellular mechanism. Since long dsRNA provokes an interferon response in mammalian cells, the technology was initially restricted to organisms or cells showing no interferon response (Bass, B. L., Nature 411, 428-429 (2001)). The finding that short (<30 bp) interfering RNAs (siRNA) circumvent the interferon response extended the application to mammalian cells (Elbashir, S. M. et al., Nature 411, 494-498 (2001)).

Although RNAi in mice has been in principle demonstrated, the current technology does not allow performing systematic gene function analysis in vivo. So far the inhibition of gene expression has been achieved by injection of purified siRNA into the tail vain of mice (McCaffrey, A. P. et al., Nature 418, 38-39 (2002); Lewis, D. H. et al., Nature Genet. 32, 107-108 (2002)). Using this approach, gene inhibition is restricted to specific organs and persists only a few days. A further improvement of the siRNA technology is based on the intracellular transcription of the two complementary siRNA strands using separate expression units both under the control of the U6 promoter (Lee, N. S. et al., 3. Nat. Biotechnol. 20(5):500-5 (2002); Du, Q. et al., Biochem. Biophys. Res. Commun. 325(1):243-9 (2004); Miyagishi, M. and Taira, K., Methods Mol. Biol.; 252:483-91 (2004)). The transgene based approach was further refined by the expression of short hairpin RNA (shRNA) molecules by a single transcription unit under the control of the U6 or H1 promoter (Brummelkamp, T. R. et al., Science 296, 550-553 (2002); Paddison, P. J. et al, Genes Dev. 16, 948-958 (2002); Yu, J. Y. et al., Proc. Natl. Acad. Sci. USA 99, 6047-6052 (2002); Sui, G. et al., Proc. Natl. Acad. Sci. USA 99, 5515-5520 (2002); Paul, C. P. et al., Nature Biotechnol. 20, 505-508 (2002); Xia, H. et al., Nat. Biotechnol. 10, 1006-10 (2002); Jacque, J. M. et al., Nature 418(6896):435-8 (2002)). The activity of shRNA in mice has been demonstrated by McCaffrey A. P. et al., Nature 418, 38-39 (2002) through injection of shRNA expression vectors into the tail vain. Again, gene inhibition was temporally and spatially restricted. Although these results demonstrate that the mechanism of shRNA mediated gene silencing is functional in mice, they do not clarify whether constitutive RNAi can be achieved in transgenic animals. Brummelkamp, T. R. et al., Science 296, 550-553 (2002), Paddison, P. J. et al., Genes Dev. 16, 948-958 (2002), Hemann, M. T. et al., Nat. Genet. 33(3):396-400 (2003); and Devroe, E. et al., BMC Biotechnol. 2(1):15 (2002) have shown the long-term inhibition of gene expression through stable integration of shRNA vectors in cultivated cell lines. These experiments included random integration of shRNA transgenes and screening for clones with appropriate siRNA expression, which is not applicable for testing of a large number of different shRNA transgenes in mice. Finally, several reports have demonstrated shRNA-mediated gene silencing in transgenic mice and rats (Hasuwa, H. et al., FEBS Lett. 532(1-2):227-30 (2002); Carmell, M. A. et al., Nat. Struct. Biol. 10(2):91-2 (2003); Rubinson, D. A. et al., Nat. Genet. 33(3):401-6 (2003); Kunath, T. et al., Nat. Biotechnol. (2003)). However, these experiments again included random integration of shRNA transgenes resulting in variable levels and patterns of shRNA expression. Thus, testing of ES cell clones or mouse lines with appropriate shRNA expression had been required, which is a laborious and time-consuming undertaking.

The in vivo validation of genes by RNAi mediated gene repression in a large scale setting requires the expression of siRNA at sufficiently high levels and with a predictable pattern in multiple organs. Targeted transgenesis provides the only approach to achieve reproducible expression of transgenes in the living organism (e.g. mammalians such as mice).

Most siRNA expression vectors are based on polymerase III dependent (Pol III) promoters (U6 or H1) that allow the production of transcripts carrying only a few non-homologous bases at their 3′ ends. It has been shown that the presence of non-homologous RNA at the ends of the shRNA stretches lower the efficiency of RNAi mediated gene silencing (Xia, H. et al., Nat. Biotechnol. 10, 1006-10 (2002)). WO 04/035782 discloses that an ubiquitous promoter driven shRNA construct provides for RNAi-mediated gene inhibition in multiple organs of the living organism. Further, an inducible gene expression system, e.g. a system based on the tetracycline dependent repressor, is suggested which allows temporary control of RNAi mediated gene silencing in transgenic cells lines and living organism. The configuration of said inducible systems as well as the choice of the repressor appeared critical with regard to the expression of inducible RNAi in multiple organs without background activity. However, since all experiments concerning inducible shRNA expression were performed in cultured cells in vitro, WO04/035782 does not allow a prediction whether such system is applicable for regulating body-wide transgene expression in a living animal (i.e. whether repression throughout development and tetracycline depend control of RNAi in different tissues does occur).

Temporary control of shRNA expression can be achieved by using engineered promoters containing a tetracycline operator (tetO) sequence (Ohkawa, J. and Taira, K., Hum. Gene Ther. 11(4):577-85 (2000)). The Tetracycline operator itself has no effect on shRNA expression. In the presence of the tetracycline repressor (tetR), however, transcription is blocked through binding of the repressor to the tetO sequence. De-repression is achieved by adding the inductor doxycycline, that causes the release of the TetR protein from the tetO site and allows transcription from the H1 promoter. Several attempts have been made to apply this strategy for the temporary control of antigens or shRNA expression in cultured cell lines (Ohkawa, J. and Taira, K., Hum. Gene Ther. 11(4):577-85 (2000); van de Wetering, M. et al., EMBO reports VOL 4, NO 6:609-615 (2003); Matsukura, 2003; Czauderna, F. et al., Nucleic Acids Res., 31(21):e127 (2003)). In these reports, the degree of doxycycline-inducible mRNA degradation was variable. In addition, background RNAi activity in the uninduced state was observed (van de Wetering, M. et al., EMBO reports VOL 4, No. 6:609-615 (2003)), indicating a limiting level of tetR expression in these cell lines.

WO 04/056964 describes the temporal control of shRNA expression in vitro using a codon-optimized tetracycline repressor. The system described in WO 04/056964 uses an engineered U6 promoter. A site-by-site comparison of the codon-optimized construct with the wildtype repressor, however, is lacking in WO 04/056964. Therefore, it is unclear whether codon optimization has any effect in the context of the particular shRNA construct used in this document. Furthermore, it is impossible to predict from the in vitro results presented in this document whether such system is applicable for regulating body-wide transgene expression in a living animal. WO 04/056964 furthermore describes the subcutaneous transplantation of transgenic cells, which were obtained by in vitro experiments, into nude mice. Again, these experiments just show the activity of shRNA constructs in a particular, transfected cell line, but not in different cell types or developmental stages of transgenic mice.

The properties of such Doxycycline-responsive promoters for siRNA expression have so far not been tested in transgenic animals. In addition, the level of shRNA expression required for efficient RNAi has never been determined and, vice versa, it is unknown whether or to which extent a basal level of shRNA expression is tolerated without significant RNAi in the uninduced state of the system. It is therefore not obvious whether a tight control of RNAi can be achieved through Doxycycline inducible expression of shRNA transgenes in living animals.

Difficulties in expression of the lac repressor and tetR in transgenic animals have been attributed to their prokaryotic origin (Scrable & Stambrook, Genetics 147:297-304 (1997); Wells, D. J., Nucleic Acids Res., 27(11):2408-15 (1999); Urlinger, S. et al., Proc. Natl. Acad. Sci. USA 97(14): 7963-8 (2000)). Alteration of the coding region by changing unfrequently used codons and eliminating putative mammalian processing signals improved the expression of these sequences (Zhang et al., Gene 105:61-72 (1991); Anastassiadis, K. et al., Gene 298:159-72 (2002)). Scrable & Stambrook, Genetics 147:297-304 (1997) were able to show expression of a codon optimized lac repressor by Northern analysis in transgenic animals, but were unable to detect protein expression and failed to prove the activity of the repressor. Anastassiadis, K. et al. demonstrated improved regulatory properties of a VP16 domain fused to a codon-optimized tet-repressor in vitro. In this system, the VP16-tetR fusion protein activates a minimal promoter through binding tet-operator sequences upon induction with doxycycline. The system therefore follows a different principle compared to transcriptional repression described in Ohkawa, J. and Taira, K., Hum. Gene Ther. 11(4):577-85 (2000); van de Wetering, M. et al., EMBO reports VOL 4, NO 6:609-615 (2003); Matsukura, 2003; Czauderna, F. et al., Nucleic Acids Res., 31(21):e127 (2003). Cronin, C. A. et al., Genes and Development 15:1506-1517 (2001) demonstrated that the expression of the lac repressor could only be achieved by an empirically combination of synthetic and wt parts of the repressor. No general prediction for transgene expression of bacterial genes in mice could be made, indicating that the codon optimization alone is not sufficient for improved transgene activity.

The provision of an inducible system allowing tight temporal control of RNAi in multicellular organisms without background activity was highly desirable.

### SUMMARY OF THE INVENTION

It was surprisingly found that a codon-optimized repressor gene, such as the tetracycline repressor gene, completely suppresses the activity of shRNA/siRNA genes under the control of a particular promoter containing the corresponding operator, such as a tetO containing promoter, in transgenic animals. In contrast thereto the same configuration with the non codon-optimized tetracycline repressor gene showed a high degree of shRNA/siRNA background activity in transgenic animals in the absence of doxycyclin induction. Thus, the present invention provides

(1) a biological entity selected from a vertebrate, a tissue culture derived from a vertebrate or one or more cells of a cell culture derived from a vertebrate, said biological entity carrying

(i) a responder construct comprising at least one segment corresponding to a short hairpin RNA (shRNA) or to complementary short interfering RNA (siRNA) strands, said at least one segment being under control of a ubiquitous promoter and said promoter containing an operator sequence being perfectly regulatable by a repressor; and

(ii) a regulator construct comprising a codon-optimized repressor gene, which provides for perfect regulation of the promoter containing the operator sequence of the responder construct;

(2) a method for preparing the biological entity as defined in (1) above or a method for constitutive and/or inducible gene knock down in a biological entity, which method comprises stably integrating

(i) a responder construct as defined in (1) above, and

(ii) a regulator construct as defined in (1) above into the genome of the biological entity; and

(3) the use of a biological entity as defined in (1) above for inducible gene knock down, and/or as a test system for pharmaceutical testing, and/or for gene target validation, and/or for gene function analysis.

### BRIEF DESCRIPTION OF THE FIGURES

FIG. 1: Principle of the Doxycycline inducible gene expression system. The tetR acts as a doxycycline-controlled transcriptional repressor. This protein binds to a modified H1-tetO sequence via the tet operator sequences in the absence of doxycycline and represses transcription.

FIG. 2: Vectors for Pol III promoter based tet-repression system (inducible): A) Insertion of a wt tetracycline repressor gene (SEQ ID NO:1) or codon-optimized tetracycline repressor gene (SEQ ID NO:2) under control of a CAGGS promoter into the rosa26 locus. B) Insertion of a shRNA containing responder construct into a ubiquitous expressed genomic locus. The transcription of the Pol II dependent Rosa26 promoter will be stopped by the synthetic polyadenylation signal (pA) and a hGH pA. An inducible Pol III promoter controls the expression of shRNA. The transcript is stopped by five thymidine bases (SEQ ID NO:3).

FIG. 3: ShRNA-mediated inhibition of luciferase expression in mice feeding doxycycline. Firefly luciferase activity in mice in the absence (black bars) or presence of H1-tetO-shRNA transgenes (uninduced: grey bars; induced through 10 days feeding with doxycycline: white bars), respectively. All mice carried the firefly and the Renilla luciferase transgenes. Relative values of Firefly luciferase activity in different organs are given as indicated. All values of Fluc activity were normalized by using the Rluc activity for reference (+/−SEM). In A all mice carried the wt tet repressor, whereas in B all mice carried the codon optimized tet repressor.

FIG. 4: Testing of IR specific shRNAs in transiently transfected C2C12 muscle cells with vectors pIR1-6. Protein extracts were analyzed two days after transfection by Western blot using an IR-specific antiserum as described in materials and methods.

FIG. 5: A) RMCE by Flp^{e} mediated recombination using the exchange vector generates the rosa26(RMCE exchanged) allele. The exchange vector carries the shRNA expression cassette under the control of the H1-tet promoter, the humanized tetR gene under the control of the CAGGS promoter, and a truncated neo^{R} gene for positive selection. A polyA signal outside the F3/FRT-flanked region is included to prevent expression of the truncated neo^{R} gene at random integration sites. The shRNA sequence for IR5 and the vector context is depicted as nucleotides. B) Southern blot analysis of genomic DNA from ES cells. The sizes of wt, rosa26(RMCE) and rosa26(RMCE exchanged) are 4.4 kb, 3.9 kb and 6.0 kb, respectively. In clones #1-3 successful RMCE had occurred. Genomic DNA was digested with HindIII and analyzed using probe 1. X: XbaI, H: HindIII. C) ES cells with (1) and without (0) the expression cassette for the shRNA against the insulin receptor were cultured in the presence of 1 μg/ml doxycycline (Dox). RNA extracts were analyzed by Northern blot using an shRNA specific antisense oligonucleotide probe.

FIG. 6: Conditional knockdown of insulin receptor expression in vivo. Three transgenic (KD1-3) and one control ES mouse (wt) were fed with 2 mg/ml doxycycline in the drinking water for 5 days. At day 6 doxycycline treated animals as well as an untreated transgenic control were sacrificed. Protein extracts prepared from various tissues were subjected to Western blot analysis using IR-specific or anti-AKT-specific antisera.

FIG. 7: Doxycycline inducible hyperglycemia in shRNA-transgenic mice. Animals where treated with 2 μg/ml (A), 20 μg/ml (B) or 2 mg/ml (C) doxycycline in the drinking water for the indicated number of days. Serum glucose levels +/− standard error of the mean are shown. All assays were performed with groups of 6 mice at age of 2 months.

FIG. 8: Reversible induction of hyperglycemia in mice. A group of six 2-month old, shIR5-transgenic mice were fed with 20 μg/ml doxycycline (Dox) in the drinking water for 10 days and subsequently kept in the absence of Dox for the next 21 days. A) Blood glucose levels were determined in venous blood samples. B) Insulin concentrations were determined on serum. Each bar represents the mean serum glucose level in six animals +/−SEM. C) Glucose tolerance test were performed on shIR5-transgenic mice before and after Dox treatment as described under methods. Results are expressed as mean blood glucose concentration +/−SEM from at least 6 animals of each group. D) Protein extracts prepared from liver were subjected to Western blot analysis using an Insr-specific antiserum or an anti-AKT-specific antiserum. Reversible knockdown of the insulin receptor using 20 μg/ml doxycycline for 10 days and 21 days after removal of Dox.

FIG. 9: A) Scheme of the targeting strategy. ShRNA and reporter constructs were independently inserted into the rosa26 locus by homologous recombination in ES cells. Genes encoding the Renilla (Rluc) and firefly luciferases (Fluc) along with an adenovirus splice acceptor sequence and a polyadenylation signal (pA) were placed downstream of the endogenous rosa26 promoter. The Fluc specific shRNA is expressed under the control of the U6-tet promoter, and terminated by five thymidines (shRNA). The loxP-sites flanking the shRNA expression cassettes were used to generate a negative control through cre-mediated recombination in ES cells. B) Southern blot analysis of genomic DNA from transfected ES cell clones containing the shRNA- (lane #1 and #2) or the reporter-constructs (lanes #3 and #4). Homologous recombination at the rosa26 locus is detectable by using EcoRV-digested genomic DNA and probe 1, resulting in a 11.7 kb band for the wt and a 2.5 kb band for targeted allele. E: EcoRV; X: XbaI; neo: FRT-flanked neomycin resistance gene; hyg: FRT-flanked hygromycin resistance gene.

FIG. 10: Efficiency of shRNA-mediated firefly luciferase (Fluc) knockdown in transgenic mice expressing the wt tetR. Each configuration (control and U6-tet shRNA) was analyzed using two to four mice at the age of 8-10 weeks, respectively. Percentages of shRNA-mediated repression of firefly luciferase activity with standard error of the mean are shown for untreated controls (gray bars) and after 10 days of feeding with 2 mg/ml doxycycline in the drinking water (white bars). In negative control animals (black bars), the shRNA expression cassettes are removed through cre-mediated recombination. Relative values of Firefly luciferase activity in different organs are shown as indicated. All values of Fluc activity were normalized by using the Rluc activity for reference.

FIG. 11: Efficiency of U6-shRNA mediated firefly luciferase (Fluc) knockdown in mice expressing the codonoptimized tet-repressor. For description see FIG. 10.

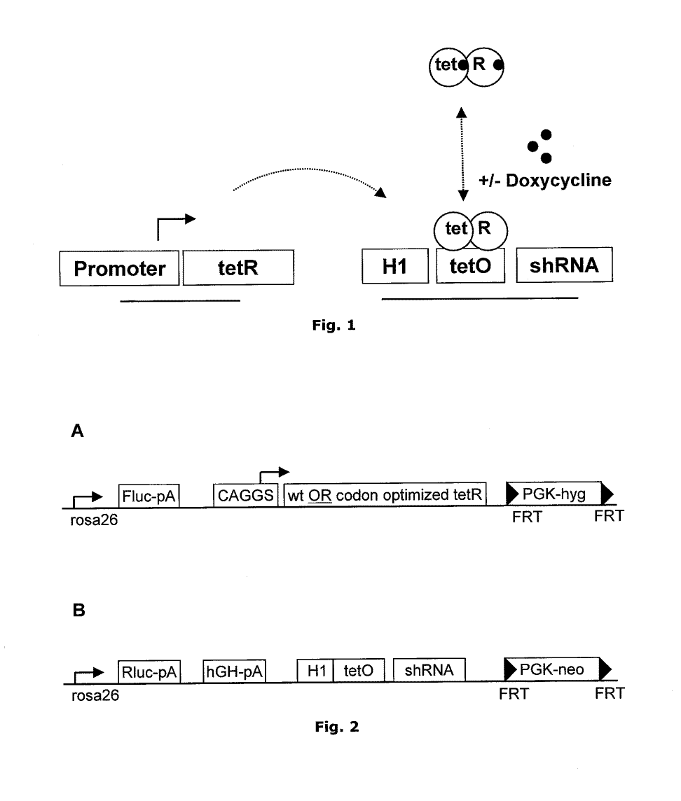

### DETAILED DESCRIPTION OF THE INVENTION

The “biological entity” according to the present invention includes, but is not limited to, a vertebrate, a tissue culture derived from a vertebrate, or one or more cells of a cell culture derived from a vertebrate.

The term “vertebrate” according to the present invention relates to multi-cellular organisms such as mammals, e.g. non-human animals such as rodents (including mice, rats, etc.) and humans, or non-mammals, e.g. fish. Most preferred vertebrates are mice and fish.

“Tissue culture” according to the present invention refers to parts of the above-defined “vertebrates” (including organs and the like) which are cultured in vitro.

“Cell culture” according to the present invention includes cells isolated from the above-defined “vertebrates” which are cultured in vitro. These cells can be transformed (immortalized) or untransformed (directly derived from vertebrates; primary cell culture).

The “responder construct” and the “regulator construct” according to the invention of the present application are suitable for stable integration into the “vertebrates” or into cells of the cell culture, e.g. by homologous recombination, recombinase mediated cassette exchange (hereinafter “RMCE”) reaction, or random integration. The vector(s) for integration of the constructs into the vertebrates by homologous recombination preferably contain(s) homologous sequences suitable for targeted integration at a defined locus, preferably at a polymerase II or III dependent locus of the living organisms or cells of the cell culture. Such polymerase II or III dependent loci include, but are not limited to, the Rosa26 locus (the murine Rosa26 locus being depicted in SEQ ID NO:11), collagen, RNA polymerase, actin, and HPRT. Homologous sequences suitable for integration into the murine Rosa26 locus are shown in SEQ ID Nos:6 and 7.

The responder construct contains at least one ubiquitous promoter which controls the expression of the at least one segment corresponding to a short hairpin RNA (shRNA) or to complementary short interfering RNA (siRNA) strands (in the following shortly referred to as “shRNA segment” and “siRNA segment”, respectively). Thus, said segment is under control of a ubiquitous promoter, wherein said promoter contains at least one operator sequence, by which said promoter is perfectly and ubiquitously regulatable by a repressor. The segment corresponding to the shRNA and siRNA are preferably comprised of DNA.

The regulator construct may also contain ubiquitous promoter(s) (constitutive, inducible or the like). Preferably the ubiquitous promoter of the regulator and/or responder construct is selected from polymerase I, II and III dependent promoters, most preferably is a polymerase II or III dependent promoter including, but not limited to, a CMV promoter, a CAGGS promoter (see nucleotides 3231-4860 of SEQ ID NO:1), a snRNA promoter such as U6, a RNAse P RNA promoter such as H1, a tRNA promoter, a 7SL RNA promoter, a 5 S rRNA promoter, etc.

The ubiquitous promoter of the “responder construct” contains an operator sequence allowing for “perfect regulation” by a corresponding repressor. “Perfect regulation” and “perfectly regulatable” within the meaning of the invention means that it permits control of the expression to an extent that no significant background activity is determined in the biological entity. This means that the suppression of the expression of the shRNA/siRNA is controlled by a rate of at least 90%, preferably by at least 95%, more preferably by at least 98%, and most preferably by 100%. Suitable operator sequences are such operator sequences, which render the promoter susceptible to regulation by the corresponding codon-optimized repressor gene present within the regulator construct, including, but not limited to, tetO, GalO, lacO, etc.

The responder construct may further contain functional sequences selected from splice acceptor sequences (such as a splice acceptor of adenovirus (see nucleotides 1129-1249 of SEQ ID NO:1), etc.), polyadenylation sites (such as synthetic polyadenylation sites (see nucleotides 2995-3173 of SEQ ID NO:1), the polyadenylation site of human growth hormones (see nucleotides 4977-5042 of SEQ ID NO:1), or the like), selectable marker sequences (such as the neomycin phosphotransferase gene of E. coli transposon, etc.), recombinase recognition sequences (such as loxP, FRT, etc), and so on.

Particularly preferred responder constructs carry a Pol III dependent promoter (inducible H1 or the like) containing tetO (for H1-tetO see nucleotides 4742-4975 of SEQ ID NO:3), and the at least one shRNA segment or siRNA segment. Particularly preferred regulator constructs carry a polymerase II (Pol II) dependent promoter (CMV, CAGGS or the like) and the codon optimized repressor gene tet.

In case shRNA segments are utilized within the responder construct, the responder construct preferably comprises at least one shRNA segment having a nucleotide (e.g. DNA) sequence of the structure A-B-C or C-B-A. In case siRNA segments are utilized within the responder construct, the responder construct preferably comprises at least two DNA segments A and C or C and A, wherein each of said at least two segments is under the control of a separate promoter as defined above (such as the Pol III promoter including inducible U6, H1 or the like). In the above segments
A is a 15 to 35, preferably a 19 to 29 bp DNA sequence being at least 90%, preferably 100% complementary to the gene to be knocked down (e.g. firefly luciferase, p53, etc.);
B is a spacer DNA sequence having 5 to 9 bp forming the loop of the expressed RNA hairpin molecule, and
C is a 15 to 35, preferably a 19 to 29 bp DNA sequence being at least 85% complementary to the sequence A.

The above shRNA and siRNA segments may further comprise stop and/or polyadenylation sequences.

Suitable siRNA sequences for the knockdown of a given target gene are well known in the art (e.g. the particular siRNA sequences mentioned in Lee N. S. et al., J. Nat. Biotechnol. 20(5):500-5 (2002) gcctgtgcctcttcagctacc (SEQ ID NO:12) and gcggagacagcgacgaagagc (SEQ ID NO:13) and in Du, Q. et al., Nucl. Acids Res. 21; 33(5):1671-7 (2005) cttattggagagagcacga (SEQ ID NO:14)) or can readily be determined by the skilled artisan.

Suitable shRNA sequences for the knock down of a given target gene are well known in the art (see e.g. the particular shRNA sequences mentioned in Tables 1 and 2 below) or can readily be determined by the skilled artisan.

TABLE 1

| target | | |
|---|---|---|
| gene | ShRNA sequence/SEQ ID NO | Reference |
| CDH-1 | TgagaagtctcccagtcagTTCAAGAGActgactgggagacttctca (19) | Brummelkamp |
| p53 | GactccagtggtaatctacTTCAAGAGAgtagattaccactggagtc (20) | et al., |
| CDC20 | CggcaggactccgggccgaTTCAAGAGAtcggcccggagtcctgccg (21) | Science, 296: |
| | | 550-3 (2002). |
| CYLD | CctcatgcagttctctttgTTCAAGAGAcaaagagaactgcatgagg (22) | Kovalenko et |
| | | al, Nature, |
| | | 424:801-5 |
| | | (2003). |
| Ras- | AagatgaagccactccctatttCAAGAGAaaatagggagtggcttcatctt (23) | Kunath et al., |
| Gap | | Nature |
| | | Biotechnology, |
| | | 21:559-561 |
| | | (2003). |
| tubulin | GacagagccaagtggactcACAgagtccacttggctctgtc (24) | Yu et al., |
| | | PNAS, 99: |
| | | 6047-52 |
| | | (2002) |
| Lamin | Ctggacttccagaagaacattcgtgttcttctggaagtccag (25) | Paul et al., |
| | | Nature Bio- |
| | | technology, |
| | | 20:505-8 |
| | | (2002). |

TABLE 2

shRNA sequences known from Brummelkamp et al., Nature,
424:797-801 (2003):

| Target | |
|---|---|
| Gene | shRNA Sequence/SEQ ID NO |
| UBIQUITIN | GAGATTGGTCCAGAACAGTTTCAAGAGAACTGTTCTGGACCAATCTC (26) |
| CARBOXYL- | GCCCTTCCGATCATGGTAGTTCAAGAGACTACCATGATCGGAAGGGC (27) |
| TERMINAL | TCTTTAGAATTCTTAAGTATTCAAGAGATACTTAAGAATTCTAAAGA (28) |
| HYDROLASE | CATTAGCTATATCAACATGTTCAAGAGACATGTTGATATAGCTAATG (29) |
| 12 | |
| UBIQUITIN | ACCACAAACGGCGGAACGATTCAAGAGATCGTTCCGCCGTTTGTGGT (30) |
| CARBOXYL- | GAGGGTCTTGGAGGTCTTCTTCAAGAGAGAAGACCTCCAAGACCCTC (31) |
| TERMINAL | GTCCATGCCCAGCCGTACATTCAAGAGATGTACGGCTGGGCATGGAC (32) |
| HYDROLASE | GCTGGACACCCTCGTGGAGTTCAAGAGACTCCACGAGGGTGTCCAGC (33) |
| 11 | |
| UBIQUITIN | GAATATCAGAGAATTGAGTTTCAAGAGAACTCAATTCTCTGATATTC (34) |
| CARBOXYL- | TGGACTTCATGAGGAAATGTTCAAGAGACATTTCCTCATGAAGTCCA (35) |
| TERMINAL | TATTGAATATCCTGTGGACTTCAAGAGAGTCCACAGGATATTCAATA (36) |
| HYDROLASE | TTGTACTGAGAGAAACTGCTTCAAGAGAGCAGTTTCTCTCAGTACAA (37) |
| 10 | |
| HAUSP | GATCAATGATAGGTTTGAATTCAAGAGATTCAAACCTATCATTGATC (38) |
| | GGAGTTTGAGAAGTTTAAATTCAAGAGATTTAAACTTCTCAAACTCC (39) |
| | GAACTCCTCGCTTGCTGAGTTCAAGAGACTCAGCAAGCGAGGAGTTC (40) |
| | CCGAATTTAACAGAGAGAATTCAAGAGATTCTCTCTGTTAAATTCGG (41) |
| UBIQUITIN | GACAGCAGAAGAATGCAGATTCAAGAGATCTGCATTCTTCTGCTGTC (42) |
| CARBOXYL- | ATAAAGCTCAACGAGAACCTTCAAGAGAGGTTCTCGTTGAGCTTTAT (43) |
| TERMINAL | GGTGAAGTGGCAGAAGAATTTCAAGAGAATTCTTCTGCCACTTCACC (44) |
| HYDROLASE | GTATTGCAGTAATCATCACTTCAAGAGAGTGATGATTACTGCAATAC (45) |
| 8 | |
| FLJ10785 | GATATGGGGTTCCATGTCATTCAAGAGATGACATGGAACCCCATATC (46) |
| | GGAGACATGGTTCTTAGTGTTCAAGAGACACTAAGAACCATGTCTCC (47) |
| | AGCACCAAGTTCGTCTCAGTTCAAGAGACTGAGACGAACTTGGTGCT (48) |
| | GATGCAACACTGAAAGAACTTCAAGAGAGTTCTTTCAGTGTTGCATC (49) |
| KIAA0710 | GTCAATGGCAGTGATGATATTCAAGAGATATCATCACTGCCATTGAC (50) |
| | CCTGCTAGCTGCCTGTGGCTTCAAGAGAGCCACAGGCAGCTAGCAGG (51) |
| | CCACCTTTGCCAGAAGGAGTTCAAGAGACTCCTTCTGGCAAAGGTGG (52) |
| | CCCTATTGAGGCAAGTGTCTTCAAGAGAGACACTTGCCTCAATAGGG (53) |
| FLJ12552/ | GAAGGAAAACTTGCTGACGTTCAAGAGACGTCAGCAAGTTTTCCTTC (54) |
| FLJ14256 | CTCACCTGGGTCCATGAGATTCAAGAGATCTCATGGACCCAGGTGAG (55) |
| | GCTGTCTTACCGTGTGGTCTTCAAGAGAGACCACACGGTAAGACAGC (56) |
| | CCTGGACCGCATGTATGACTTCAAGAGAGTCATACATGCGGTCCAGG (57) |
| KIAA1203 | GTCAATGGCAGTGATGATATTCAAGAGATATCATCACTGCCATTGAC (58) |
| | CCTGCTAGCTGCCTGTGGCTTCAAGAGAGCCACAGGCAGCTAGCAGG (59) |
| | CCACCTTTGCCAGAAGGAGTTCAAGAGACTCCTTCTGGCAAAGGTGG (60) |
| | CCCTATTGAGGCAAGTGTCTTCAAGAGAGACACTTGCCTCAATAGGG (61) |
| FLJ23277 | GGAAATCCGAATTGCTTGGTTCAAGAGACCAAGCAATTCGGATTTCC (62) |
| | CACATTTCTTCAAGTGTGGTTCAAGAGACCACACTTGAAGAAATGTG (63) |
| | CAGCAGGATGCTCAAGAATTTCAAGAGAATTCTTGAGCATCCTGCTG (64) |
| | GCTGAATACCTACATTGGCTTCAAGAGAGCCAATGTAGGTATTCAGC (65) |
| FLJ14914 | GGGCTTGTGCCTGGCCTTGTTCAAGAGACAAGGCCAGGCACAAGCCC (66) |
| (similar | GCCTTGTCCTGCCAAGAAGTTCAAGAGACTTCTTGGCAGGACAAGGC (67) |
| to UBP4) | GATTGAAGCCAAGGGAACGTTCAAGAGACGTTCCCTTGGCTTCAATC (68) |
| | TGGCGCCTGCTCCCCATCTTTCAAGAGAAGATGGGGAGCAGGCGCCA (69) |
| UBIQUITIN | GAACCAGCAGGCTCTGTGGTTCAAGAGACCACAGAGCCTGCTGGTTC (70) |
| CARBOXYL- | GGAAGCATAATTATCTGCCTTCAAGAGAGGCAGATAATTATGCTTCC (71) |
| TERMINAL | AGAAGAAGATGCTTTTCACTTCAAGAGAGTGAAAAGCATCTTCTTCT (72) |
| HYDROLASE | CTTGCAGAGGAGGAACCCATTCAAGAGATGGGTTCCTCCTCTGCAAG (73) |
| ISOZYME L5 | |
| UBIQUITIN | GCAAACAATCAGCAATGCCTTCAAGAGAGGCATTGCTGATTGTTTGC (74) |
| CARBOXYL- | TTGGACTGATTCATGCTATTTCAAGAGAATAGCATGAATCAGTCCAA (75) |
| TERMINAL | CTGGCAATTCGTTGATGTATTCAAGAGATACATCAACGAATTGCCAG (76) |
| HYDROLASE | TTAGATGGGCGGAAGCCATTTCAAGAGAATGGCTTCCGCCCATCTAA (77) |
| ISOZYME L3 | |
| UBIQUITIN | GAGGAGTCTCTGGGCTCGGTTCAAGAGACCGAGCCCAGAGACTCCTC (78) |
| CARBOXYL- | GAGCTGAAGGGACAAGAAGTTCAAGAGACTTCTTGTCCCTTCAGCTC (79) |
| TERMINAL | TGTCGGGTAGATGACAAGGTTCAAGAGACCTTGTCATCTACCCGACA (80) |
| HYDROLASE | CACAGCTGTTCTTCTGTTCTTCAAGAGAGAACAGAAGAACAGCTGTG (81) |
| ISOZYME L1 | |
| KIAA1891/ | GTGGAAGCCTTTACAGATCTTCAAGAGAGATCTGTAAAGGCTTCCAC (82) |
| FLJ25263 | CAACAGCTGCCTTCATCTGTTCAAGAGACAGATGAAGGCAGCTGTTG (83) |
| | CCATAGGCAGTCCTCCTAATTCAAGAGATTAGGAGGACTGCCTATGG (84) |
| | TGTATCACTGCCACTGGTTTTCAAGAGAAACCAGTGGCAGTGATACA (85) |
| FLJ14528 | CATGTTGGGCAGCTGCAGCTTCAAGAGAGCTGCAGCTGCCCAACATG (86) |
| (similar | CACAACTGGAGACCTGAAGTTCAAGAGACTTCAGGTCTCCAGTTGTG (87) |
| to UBP8) | GTATGCCTCCAAGAAAGAGTTCAAGAGACTCTTTCTTGGAGGCATAC (88) |
| | CTTCACAGTACATTTCTCTTTCAAGAGAAGAGAAATGTACTGTGAAG (89) |
| U4/U6 TRI | GTACTTTCAAGGCCGGGGTTTCAAGAGAACCCCGGCCTTGAAAGTAC (90) |
| SNRNP 65 | CTTGGACAAGCAAGCCAAATTCAAGAGATTTGGCTTGCTTGTCCAAG (91) |
| kDa protein | GACTATTGTGACTGATGTTTTCAAGAGAAACATCAGTCACAATAGTC (92) |
| | GGAGAACTTTCTGAAGCGCTTCAAGAGAGCGCTTCAGAAAGTTCTCC (93) |
| XM_089437 | GACGAGAGAAACCTTCACCTTCAAGAGAGGTGAAGGTTTCTCTCGTC (94) |
| | ACATTATTCTACATTCTTTTTCAAGAGAAAAGAATGTAGAATAATGT (95) |
| | AGATTCGCAAATGGATGTATTCAAGAGATACATCCATTTGCGAATCT (96) |
| | CATTCCCACCATGAGTCTGTTCAAGAGACAGACTCATGGTGGGAATG (97) |
| KIAA1453 | GATCGCCCGACACTTCCGCTTCAAGAGAGCGGAAGTGTCGGGCGATC (98) |
| | CCAGCAGGCCTACGTGCTGTTCAAGAGACAGCACGTAGGCCTGCTGG (99) |
| | GCCAGCTCCTCCACAGCACTTCAAGAGAGTGCTGTGGAGGAGCTGGC (100) |
| | CGCCGCCAAGTGGAGCAGATTCAAGAGATCTGCTCCACTTGGCGGCG (101) |
| FLJ12697 | GAAGATGCCCATGAATTCCTTCAAGAGAGGAATTCATGGGCATCTTC (102) |
| | CAAACAGGCTGCGCCAGGCTTCAAGAGAGCCTGGCGCAGCCTGTTTG (103) |
| | ACGGCCTAGCGCCTGATGGTTCAAGAGACCATCAGGCGCTAGGCCGT (104) |
| | CTGTAACCTCTCTGATCGGTTCAAGAGACCGATCAGAGAGGTTACAG (105) |
| UBIQUITIN | TCTGTCAGTCCATCCTGGCTTCAAGAGAGCCAGGATGGACTGACAGA (106) |
| SPECIFIC | TGAAGCGAGAGTCTTGTGATTCAAGAGATCACAAGACTCTCGCTTCA (107) |
| PROTEASE | GATGGAGTGCTAATGGAAATTCAAGAGATTTCCATTAGCACTCCATC (108) |
| 18 (USP18) | CCTTCAGAGATTGACACGCTTCAAGAGAGCGTGTCAATCTCTGAAGG (109) |
| UBIQUITIN | CCTGACCACGTTCCGACTGTTCAAGAGACAGTCGGAACGTGGTCAGG (110) |
| CARBOXYL- | GAGTTCCTTCGCTGCCTGATTCAAGAGATCAGGCAGCGAAGGAACTC (111) |
| TERMINAL | GACTGCCTTGCTGCCTTCTTTCAAGAGAAGAAGGCAGCAAGGCAGTC (112) |
| HYDROLASE | CGCCGAGGGCTACGTACTCTTCAAGAGAGAGTACGTAGCCCTCGGCG (113) |
| 20 | |
| UBIQUITIN | GGCGAGAAGAAAGGACTGTTTCAAGAGAACAGTCCTTTCTTCTCGCC (114) |
| CARBOXYL- | GGACGAGAATTGATAAAGATTCAAGAGATCTTTATCAATTCTCGTCC (115) |
| TERMINAL | GCACGAGAATTTGGGAATCTTCAAGAGAGATTCCCAAATTCTCGTGC (116) |
| HYDROLASE | CTACTTCATGAAATATTGGTTCAAGAGACCAATATTTCATGAAGTAG (117) |
| 24 | |
| KIAA1594 | GATAACAGCTTCTTGTCTATTCAAGAGATAGACAAGAAGCTGTTATC (118) |
| | GAGAATAGGACATCAGGGCTTCAAGAGAGCCCTGATGTCCTATTCTC (119) |
| | CTTGGAAGACTGAACCTGTTTCAAGAGAACAGGTTCAGTCTTCCAAG (120) |
| | CAACTCCTTTGTGGATGCATTCAAGAGATGCATCCACAAAGGAGTTG (121) |
| KIAA1350 | GATGTTGTCTCCAAATGCATTCAAGAGATGCATTTGGAGACAACATC (122) |
| | CGTGGGGACTGTACCTCCCTTCAAGAGAGGGAGGTACAGTCCCCACG (123) |
| | GTACAGCTTCAGAACCAAGTTCAAGAGACTTGGTTCTGAAGCTGTAC (124) |
| UBIQUITIN | GATGATCTTCAGAGAGCAATTCAAGAGATTGCTCTCTGAAGATCATC (125) |
| CARBOXYL- | GGAACATCGGAATTTGCCTTTCAAGAGAAGGCAAATTCCGATGTTCC (126) |
| TERMINAL | GAGCTAGTGAGGGACTCTTTTCAAGAGAAAGAGTCCCTCACTAGCTC (127) |
| HYDROLASE | GCAGGGTTCTTTAAGGCAATTCAAGAGATTGCCTTAAAGAACCCTGC (128) |
| 25 | |
| UBIQUITIN | TCGATGATTCCTCTGAAACTTCAAGAGAGTTTCAGAGGAATCATCGA (129) |
| CARBOXYL- | GATAATGGAAATATTGAACTTCAAGAGAGTTCAATATTTCCATTATC (130) |
| TERMINAL | GTTCTTCATTTAAATGATATTCAAGAGATATCATTTAAATGAAGAAC (131) |
| HYDROLASE | GTTAACAAACACATAAAGTTTCAAGAGAACTTTATGTGTTTGTTAAC (132) |
| 16 | |
| USP9X | GTTAGAGAAGATTCTTCGTTTCAAGAGAACGAAGAATCTTCTCTAAC (133) |
| | GTTGATTGGACAATTAAACTTCAAGAGAGTTTAATTGTCCAATCAAC (134) |
| | GGTTGATACCGTAAAGCGCTTCAAGAGAGCGCTTTACGGTATCAACC (135) |
| | GCAATGAAACGTCCAATGGTTCAAGAGACCATTGGACGTTTCATTGC (136) |
| USP9Y | AGCTAGAGAAAATTCTTCGTTCAAGAGACGAAGAATTTTCTCTAGCT (137) |
| | GATCCTATATGATGGATGATTCAAGAGATCATCCATCATATAGGATC (138) |
| | GTTCTTCTTGTCAGTGAAATTCAAGAGATTTCACTGACAAGAAGAAC (139) |
| | CTTGAGCTTGAGTGACCACTTCAAGAGAGTGGTCACTCAAGCTCAAG (140) |
| UBIQUITIN | GACCGGCCAGCGAGTCTACTTCAAGAGAGTAGACTCGCTGGCCGGTC (141) |
| CARBOXYL- | GGACCTGGGCTACATCTACTTCAAGAGAGTAGATGTAGCCCAGGTCC (142) |
| TERMINAL | CTCTGTGGTCCAGGTGCTCTTCAAGAGAGAGCACCTGGACCACAGAG (143) |
| HYDROLASE | GACCACACGATTTGCCTCATTCAAGAGATGAGGCAAATCGTGTGGTC (144) |
| 5 | |
| UBIQUITIN | TGGCTTGTTTATTGAAGGATTCAAGAGATCCTTCAATAAACAAGCCA (145) |
| CARBOXYL- | GTGAATTTGGGGAAGATAATTCAAGAGATTATCTTCCCCAAATTCAC (146) |
| TERMINAL | CGCTATAGCTTGAATGAGTTTCAAGAGAACTCATTCAAGCTATAGCG (147) |
| HYDROLASE | GATATCCTGGCTCCACACATTCAAGAGATGTGTGGAGCCAGGATATC (148) |
| 26 | |
| KIAA1097 | GAGCCAGTCGGATGTAGATTTCAAGAGAATCTACATCCGACTGGCTC (149) |
| | GTAAATTCTGAAGGCGAATTTCAAGAGAATTCGCCTTCAGAATTTAC (150) |
| | GCCCTCCTAAATCAGGCAATTCAAGAGATTGCCTGATTTAGGAGGGC (151) |
| | GTTGAGAAATGGAGTGAAGTTCAAGAGACTTCACTCCATTTCTCAAC (152) |
| UBIQUITIN | GCTTGGAAAATGCAAGGCGTTCAAGAGACGCCTTGCATTTTCCAAGC (153) |
| SPECIFIC | CTGCATCATAGACCAGATCTTCAAGAGAGATCTGGTCTATGATGCAG (154) |
| PROTEASE | GATCACCACGTATGTGTCCTTCAAGAGAGGACACATACGTGGTGATC (155) |
| 22 (USP22) | TGACAACAAGTATTCCCTGTTCAAGAGACAGGGAATACTTGTTGTCA (156) |
| UBIQUITIN- | GAAATATAAGACAGATTCCTTCAAGAGAGGAATCTGTCTTATATTTC (157) |
| SPECIFIC | CCCATCAAGTTTAGAGGATTTCAAGAGAATCCTCTAAACTTGATGGG (158) |
| PROCESSING | GGTGTCCCATGGGAATATATTCAAGAGATATATTCCCATGGGACACC (159) |
| PROTEASE 29 | GAATGCCGACCTACAAAGATTCAAGAGATCTTTGTAGGTCGGCATTC (160) |
| CYLD | CAGTTATATTCTGTGATGTTTCAAGAGAACATCACAGAATATAACTG (161) |
| | GAGGTGTTGGGGACAAAGGTTCAAGAGACCTTTGTCCCCAACACCTC (162) |
| | GTGGGCTCATTGGCTGAAGTTCAAGAGACTTCAGCCAATGAGCCCAC (163) |
| | GAGCTACTGAGGACAGAAATTCAAGAGATTTCTGTCCTCAGTAGCTC (164) |
| UBIQUITIN | TCAGCAGGATGCTCAGGAGTTCAAGAGACTCCTGAGCATCCTGCTGA (165) |
| CARBOXYL- | GAAGTTCTCCATCCAGAGGTTCAAGAGACCTCTGGATGGAGAACTTC (166) |
| TERMINAL | GCCGGTCCCCACCAGCAGCTTCAAGAGAGCTGCTGGTGGGGACCGGC (167) |
| HYDROLASE 2 | CACTCGGGAGTTGAGAGATTTCAAGAGAATCTCTCAACTCCCGAGTG (168) |
| UBIQUITIN | GCCCTTGGGTCTGTTTGACTTCAAGAGAGTCAAACAGACCCAAGGGC (169) |
| SPECIFIC | CTCAACACTAAACAGCAAGTTCAAGAGACTTGCTGTTTAGTGTTGAG (170) |
| PROTEASE 3 | GATTTCATTGGACAGCATATTCAAGAGATATGCTGTCCAATGAAATC (171) |
| (USP3) | CATGGGGCACCAACTAATTTTCAAGAGAAATTAGTTGGTGCCCCATG (172) |
| UBIQUITIN | GGTGTCTCTGCGGGATTGTTTCAAGAGAACAATCCCGCAGAGACACC (173) |
| CARBOXYL- | AGTTCAGTAGGTGTAGACTTTCAAGAGAAGTCTACACCTACTGAACT (174) |
| TERMINAL | GAGTTCCTGAAGCTCCTCATTCAAGAGATGAGGAGCTTCAGGAACTC (175) |
| HYDROLASE | GGATTTGCTGGGGGCAAGGTTCAAGAGACCTTGCCCCCAGCAAATCC (176) |
| 23 | |
| UBP-32.7 | CTCAGAAAGCCAACATTCATTCAAGAGATGAATGTTGGCTTTCTGAG (177) |
| | CGCATTGTAATAAGAAGGTTTCAAGAGAACCTTCTTATTACAATGCG (178) |
| | GGGAGGAAAATGCAGAAATTTCAAGAGAATTTCTGCATTTTCCTCCC (179) |
| | TTACAAATTTAGGAAATACTTCAAGAGAGTATTTCCTAAATTTGTAA (180) |
| HOMO SAPIENS | GTTATGAATTGATATGCAGTTCAAGAGACTGCATATCAATTCATAAC (181) |
| UBIQUITIN | GTGATAACACAACTAATGGTTCAAGAGACCATTAGTTGTGTTATCAC (182) |
| SPECIFIC | GTAGAGGAGAGTTCTGAAATTCAAGAGATTTCAGAACTCTCCTCTAC (183) |
| PROTEASE 13 | GCCTCTAATCCTGATAAGGTTCAAGAGACCTTATCAGGATTAGAGGC (184) |
| ISOPEPTIDASE | |
| T-3) | |
| UBIQUITIN | GATGATCTTCAGGCTGCCATTCAAGAGATGGCAGCCTGAAGATCATC (185) |
| CARBOXYL- | GTATGGACAAGAGCGTTGGTTCAAGAGACCAACGCTCTTGTCCATAC (186) |
| TERMINAL | CGAACCCTTCTGGAACAGTTTCAAGAGAACTGTTCCAGAAGGGTTCG (187) |
| HYDROLASE | GTGGCATGAAGATTATAGTITCAAGAGAACTATAATCTTCATGCCAC (188) |
| 28 | |
| UBIQUITIN | GGTGAACAAGGACAGTATCTTCAAGAGAGATACTGTCCTTGTTCACC (189) |
| CARBOXYL- | GCAATAGAGGATGATTCTGTTCAAGAGACAGAATCATCCTCTATTGC (190) |
| TERMINAL | TCTGTGAATGCCAAAGTTCTTCAAGAGAGAACTTTGGCATTCACAGA (191) |
| HYDROLASE | CACACCAGGGAAGGTCTAGTTCAAGAGACTAGACCTTCCCTGGTGTG (192) |
| 14 | |
| DUB1 | GCAGGAAGATGCCCATGAATTCAAGAGATTCATGGGCATCTTCCTGC (193) |
| | GAATGTGCAATATCCTGAGTTCAAGAGACTCAGGATATTGCACATTC (194) |
| | TGGATGATGCCAAGGTCACTTCAAGAGAGTGACCTTGGCATCATCCA (195) |
| | GCTCCGTGCTAAACCTCTCTTCAAGAGAGAGAGGTTTAGCACGGAGC (196) |
| MOUSE | GCCTCCACCTCAACAGAGGTTCAAGAGACCTCTGTTGAGGTGGAGGC (197) |
| USP27 | CTGCATCATAGACCAAATCTTCAAGAGAGATTTGGTCTATGATGCAG (198) |
| HOMOLOG | GATCACTACATACATTTCCTTCAAGAGAGGAAATGTATGTAGTGATC (199) |
| | GTAAAGAGAGCAGAATGAATTCAAGAGATTCATTCTGCTCTCTTTAC (200) |
| UBIQUITIN | CGCGGGGCGCAGTGGTATCTTCAAGAGAGATACCACTGCGCCCCGCG (201) |
| CARBOXYL- | CAGAAGGCAGTGGGGAAGATTCAAGAGATCTTCCCCACTGCCTTCTG (202) |
| TERMINAL | GCCTGGGAGAATCACAGGTTTCAAGAGAACCTGTGATTCTCCCAGGC (203) |
| HYDROLASE 4 | ACCAGACAAGGAAATACCCTTCAAGAGAGGGTATTTCCTTGTCTGGT (204) |
| TRE-2 | CACATCCACCACATCGACCTTCAAGAGAGGTCGATGTGGTGGATGTG (205) |
| | GTCACAACCCAAGACCATGTTCAAGAGACATGGTCTTGGGTTGTGAC (206) |
| | CTCAACAGGACAAATCCCATTCAAGAGATGGGATTTGTCCTGTTGAG (207) |
| | TAGATCAATTATTGTGGATTTCAAGAGAATCCACAATAATTGATCTA (208) |
| UBIQUITIN | GGAACACCTTATTGATGAATTCAAGAGATTCATCAATAAGGTGTTCC (209) |
| CARBOXYL- | CTTTAACAGAAATTGTCTCTTCAAGAGAGAGACAATTTCTGTTAAAG (210) |
| TERMINAL | CCTATGCAGTACAAAGTGGTTCAAGAGACCACTTTGTACTGCATAGG (211) |
| HYDROLASE | GATCTTTTCTTGCTTTGGATTCAAGAGATCCAAAGCAAGAAAAGATC (212) |
| 15 (UNPH-2). | |
| KIAA1372 | CAGCATCCTTCAGGCCTTATTCAAGAGATAAGGCCTGAAGGATGCTG (213) |
| | GATAGTGACTCGGATCTGCTTCAAGAGAGCAGATCCGAGTCACTATC (214) |
| | GACATCACAGCCCGGGAGTTTCAAGAGAACTCCCGGGCTGTGATGTC (215) |
| | GGACACAGCCTATGTGCTGTTCAAGAGACAGCACATAGGCTGTGTCC (216) |
| BRCA1 | GTGGAGGAGATCTACGACCTTCAAGAGAGGTCGTAGATCTCCTCCAC (217) |
| ASSOCIATED | CTCTTGTGCAACTCATGCCTTCAAGAGAGGCATGAGTTGCACAAGAG (218) |
| PROTEIN-1 | ACAGGGCCCCTGCAGCCTCTTCAAGAGAGAGGCTGCAGGGGCCCTGT (219) |
| | GAAGACCTGGCGGCAGGTGTTCAAGAGACACCTGCCGCCAGGTCTTC (220) |

The “regulator construct” comprises a repressor gene, which provides for perfect regulation of the operators of the responder construct. In particular, the repressor gene encodes a repressor, i.e. a molecule acting on the operator of the promoter to therewith inhibit (down-regulate) the expression of the shRNA/siRNA. Suitable repressor genes include codon-optimized repressors (i.e., repressor genes where the codon usage is adapted to the codon usage of vertebrates), including, but not limited to, a codon-optimized tet repressor, a codon-optimized Gal repressor, a codon-optimized lac repressor and variants thereof. Particularly preferred is the codon optimized tet repressor, most preferred a codon-optimized tet repressor having the sequence of nucleotides 5149 to 5916 of SEQ ID NOs:2 or 3.

Embodiment (2) of the invention pertains to a method for preparing the biological entity as defined hereinbefore and to a method for constitutive and/or inducible gene knock down in a biological entity, which stably integrating

(i) the responder construct as defined hereinbefore, and

(ii) a regulator construct as defined hereinbefore

into the genome of the biological entity.

In particular the method comprises subsequent or contemporary integration of the responder construct, and the regulator construct into the genome of vertebrate cells. In case of (non-human) mammals the constructs are preferably integrated into embryonic stem (ES) cells of said mammals.

Various methods are applicable for the integration of the constructs.

A first integration method is the so called “homologous recombination” which utilizes an integration vector comprising the functional nucleotide sequence to be integrated and DNA sequences homologous to the integration site, where said homologous DNA sequences flank the functional nucleotide sequence. In a particular preferred embodiment of the invention, both, the responder construct and the regulator construct are integrated by homologous recombination on the same or different allel(s).

A second integration method is the RMCE reaction, which comprises the steps of

(i) modifying a starting cell by introducing an acceptor DNA which integrates into the genome of the starting cell (e.g. by homologous recombination), and wherein the acceptor DNA comprises two mutually incompatible recombinase recognition sites (RRSs), and introducing into such modified cell;

(ii) a donor DNA comprising the same two mutually incompatible RRSs contained in the acceptor DNA by utilizing an integration vector comprising a functional DNA sequence flanked by the RRSs; and

(iii) a recombinase which catalyzes recombination between the RRSs of the acceptor and donor.

In a preferred embodiment of the invention the integration of at least one of the responder construct and the regulator construct is effected by RMCE reaction.

Details of the first and second method, in particular for integration at the murine Rosa26 locus are discussed in detail in applicant's WO 2004/063381, the disclosure of which is herewith incorporated by reference. For the integration at the murine Rosa26 locus (the sequence thereof being depicted in SEQ ID NO:11) by homologous recombination, the integration vector caries homologous flanking sequences of 0.2 to 20 kB, preferably 1 to 8 kB length. Suitable sequences include, but are not limited to, the sequences depicted in SEQ ID NOs:6 and 7.

A third integration method is the so-called “random transgenesis” where an integration vector is randomly integrated into the genome of the cell. By pronucleus injection of the linearized vector one or more copies of the DNA-fragment integrates randomly into the genome of the mouse embryo. The resulting founder lines have to be characterized for the expression of the transgene (Palmiter, R. D. and Brinster, R. L., Annu. Rev. Genet. 20:465-499 (1986)). Hasuwa H. et al. FEBS Lett. 532(1-2):227-230 (2002) used this technology for the generation of siRNA expressing mice and rats.

Particularly preferred in the invention is that the integration vector (in all three integration methods discussed above) carries both, the responder construct and the regulator construct.

The preparation of the vertebrate is hereinafter further described by reference to the mouse system. This shall, however, not be construed as limiting the invention. The preferred method for producing a shRNA in a mouse (and also mouse tissue and cells derived from such mouse) that expresses the codon optimized repressor protein comprising the steps of:
(i) insertion of a repressor construct carrying a codon-optimized repressor gene, such as the tet repressor gene, into the mouse genome; and
(ii) insertion of a responder construct containing
   one or more promoter sequence(s), each carrying at least one operator sequence (such as tetO, etc.) positioned 1 to 10 bp, preferably 1 to 2 bp 3′ and/or 5′ of the TATA element and
   a DNA sequence encoding a shRNA or siRNA as defined hereinbefore lying 3′ to the said at least one operator sequence
   into the mouse genome; and
(iii) generation of mice from steps (i) and (ii); or
(iv) generation of mice from step (i) and generation of mice from step (ii) and a subsequent breeding of these two lines.

The inducible gene knock-down according to embodiments (2) and (3) of the invention moreover comprises the step of administering a suitable inducer compound to the biological entity (in particular the vertebrate) or ceasing the administering of the inducer compound to therewith induce or cease the expression of the respective siRNA.

The technology of the present application provides for the following advantages:

(i) a stable and body wide inhibition of gene expression by generating transgenic animals (such as mice);

(ii) a reversible inhibition of gene expression using the inducible constructs.

The invention is furthermore described by the following examples which are, however, not to be construed so as to limit the invention.

### EXAMPLES

### Example 1

Plasmid construction: All plasmid constructs were generated by standard DNA cloning methods.

Basic rosa26 targeting vector: A 129 SV/EV-BAC library (Incyte Genomics) was screened using a probe against exon2 of the Rosa26 locus (amplified from mouse genomic DNA using Rscreen1s (GACAGGACAGTGCTTGTTTAAGG; SEQ ID NO:4) and Rscreen1as (TGACTACACAATATTGCTCGCAC; SEQ ID NO:5)). Out of the identified BACclone a 11 kb EcoRV subfragment was inserted into the HindIII site of pBS. Two fragments (a 1 kb SacII/XbaI- and a 4 kb XbaI-fragment; see SEQ ID NOs:6 and 7) were used as homology arms and inserted into a vector containing a FRT-flanked neomycin resistance gene or hygromycin resistance gene to generate the basic Rosa26 targeting vectors. The splice acceptor site (SA) from adenovirus (Friedrich, G. and Soriano, P., Genes Dev., 5:1513-23 (1991)) was inserted as PCR-fragment (amplified using the oligonucleotides ATACCTGCAGGGGTGACCTGCACGTCTAGG (SEQ ID NO:15) and ATACCTGCAGGAGTACTGGAAAGACCGCGAAG (SEQ ID NO:16)) between the 5′ arm and the FRT flanked neomycin resistance gene or the FRT flanked hygromycin resistant gene. The Renilla luciferase (Rluc) and firefly luciferase (Fluc) coding regions (Promega) were placed 3′ of the SA site (Friedrich, G. and Soriano, P., Genes Dev. 9:1513-23 (1991); see SEQ ID NOs:1, 2 and 3)) to facilitate transcription from the endogenous rosa26 promoter.

Insertion of transgenes into the targeting vector: All subsequently described transgenes were inserted 3′ of the Renilla luciferase (Rluc) or firefly luciferase genes. The H1-promoter fragments were amplified from human genomic DNA (using the oligonucleotides AACTATGGCCGGCCGAAGAACTCGTCAAGAAGGCG (SEQ ID NO: 17) and TATGGTACCGTTTAAACGCGGCCGCAAATTTATTAGAGC (SEQ ID NO:18)) and the tet-operator sequences was placed 3′ of the TATA-box. 3′ of the H1-promoter with the tet-operator sequence a Fluc-specific shRNA was inserted by BbsI/AscI using annealed oligonucleotides forming the sequence aggattccaattcagcgggagccacct gatgaagcttgatcgggtggctctcgctgagttggaatccattttttt (SEQ ID NO:8; Paddison, P. J. et al., Genes Dev. 16:948-58 (2002)). The codon optimized tet-repressor was PCR amplified from pBS-hTA+nls (Anastassiadis, K. et al., Gene 298:159-72 (2002)) using the oligonucleotides atcgaattcaccatgtccagactgg (sense; SEQ ID NO:9), ataggatccttaagagccagactca catttcagc (antisense; SEQ ID NO:10)) and inserted 3′ of the CAGGS promoter.

Vector 1 (SEQ ID NO: 1) contains the following elements in 5′ to 3′ orientation: 5′ homology region for murine rosa26 locus (nucleotides 24-1079), adenovirus splice acceptor site (nucleotides 1129-1249), firefly luciferase (nucleotides 1325-2977), synthetic polyA (2995-3173), CAGGS promoter (nucleotides 3231-4860), synthetic intron (nucleotides 4862-5091), coding region of the wt tet repressor (nucleotides 5148-5750), synthetic polyA (nucleotides 5782-5960), FRT-site (nucleotides 6047-6094), PGK-hygro-polyA (nucleotides 6114-8169), FRT-site, 3′ homology region for rosa26 locus (nucleotides 8312-12643), PGK-Tk-polyA (nucleotides 12664-14848).

Vector 2 (SEQ ID NO:2) contains the following elements in 5′ to 3′ orientation: 5′ homology region for rosa26 locus (nucleotides 24-1102), adenovirus splice acceptor site (nucleotides 1129-1249), firefly luciferase (nucleotides 1325-2977), synthetic polyA (nucleotides 2995-3173), CAGGS promoter (nucleotides 3231-4860), synthetic intron (nucleotides 4862-5091), coding region of the codon optimized tet repressor (nucleotides 5149-5916), synthetic polyA (nucleotides 5946-6124), FRT-site (nucleotides 6211-6258), PGK-hygro-polyA (nucleotides 6278-8333), FRT-site, 3′ homology region for rosa26 locus (nucleotides 8476-12807), PGK-Tk-polyA (nucleotides 12828-15012).

Vector 3 (SEQ ID NO:3) contains the following elements in 5′ to 3′ orientation: 5′ homology region for rosa26 locus (nucleotides 31-2359), adenovirus splice acceptor site (nucleotides 2409-2529), Renilla luciferase (nucleotides 2605-3540), synthetic polyA (nucleotides 3558-3736), hgH-polyA (nucleotides 3769-4566), loxP-site (nucleotides 4587-4620), H1-tetO (nucleotides 4742-4975), shRNA (nucleotides 4977-5042), TTTTTT, loxP-site (nucleotides 5056-5089), FRT-site (nucleotides 5105-5152), PGK-hygro-polyA (nucleotides 5165-6974), FRT-site (nucleotides 6982-7029), 3′ homology region for rosa26 locus (nucleotides 7042-11373), PGK-Tk-polyA (nucleotides 11394-13578).

Cell culture: Culture and targeted mutagenesis of ES cells were carried out as described in Hogan, B. et al., A Laboratory Manual. In Manipulating the Mouse Embryo. Cold Spring Harbor Laboratory Press, Cold Spring Harbor N.Y., pp. 253-289 (1994) with ES cell lines derived from F1 embryos. Cre-mediated deletion has been performed for the deletion of the shRNA part of the constructs to generate the control mice without knockdown. Therefore 5 μg of a cre-expressing construct has been electroporated and the following day 1000 cells were plated at a 10 cm dish. The developing clones were isolated and screened by southern for cre-mediated deletion of the shRNA responder construct.

Generation of chimeric mice: Recombinant ES cells were injected into blastocysts from Balb/C mice and chimeric mice were obtained upon transfer of blastocysts into pseudo-pregnant females using standard protocols (Hogan, B. et al. Manipulating the Mouse Embryo: A Laboratory Manual. Cold Spring Harbor Laboratory Press, Cold Spring Harbor N.Y. 253-289 (1994)).

Preparation and application of doxycycline: 2 mg doxycycline (Sigma, D-9891) was solved in 1 liter H₂O with 10% Sucrose. This solution was given in drinking bottles of mice and prepared freshly every 3 days.

Luciferase measurement in organs: Organs were homogenized at 4° C. in lysis buffer (0.1 M KH₂PO₄, 1 mM DTT, 0.1% Triton® X-100) using a tissue grinder. Spin for 5 min at 2000×g (4° C.) to pellet debris and assay supernatant for Luc activities using the Dual Luciferase Assay (Promega, Inc.) according to the manufacturer protocol.

Discussion: The coding regions of the wt (Gossen and Bujard, PNAS. 89: 5547-5551; FIG. 2; SEQ ID NO:1) or the codon optimized tet repressor (Anastassiadis, K. et al., Gene 298:159-72 (2002)) under control of the strong CAGGS promoter along with a hygromycine resistance gene and a firefly luciferase gene were inserted into the first allele of rosa26 by homologous recombination in ES cells (FIG. 2A; SEQ ID NO:2). The shRNA coding region under the control of the H1 promoter containing tet-operator sequences (H1-tetO), along with a Renilla luciferase gene and a neomycin resistance gene for positive selection of recombinant clones was inserted into the second allele of the rosa26 locus (FIG. 2B; SEQ ID NO:3). To examine the activity of the Rosa26 and H1-tetO-shRNA transgenes in vivo, recombinant ES cells of the three independent constructs described above (SEQ ID NOs:1 to 3) were injected into diploid blastocysts and chimeric mice were obtained upon transfer of blastocysts into pseudopregnant females. Mice were bred to generate double transgenic animals containing the constructs shown in SEQ ID NOs:1 and 3 or SEQ ID NOs:2 and 3, respectively.

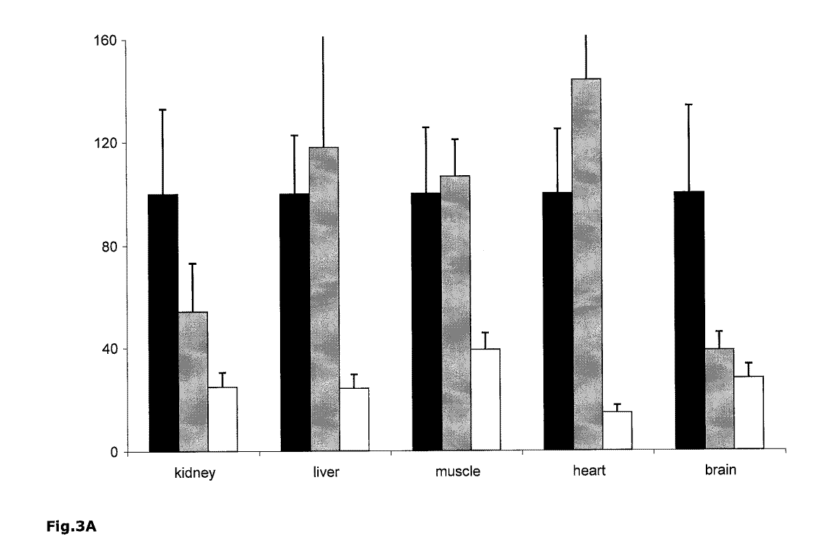

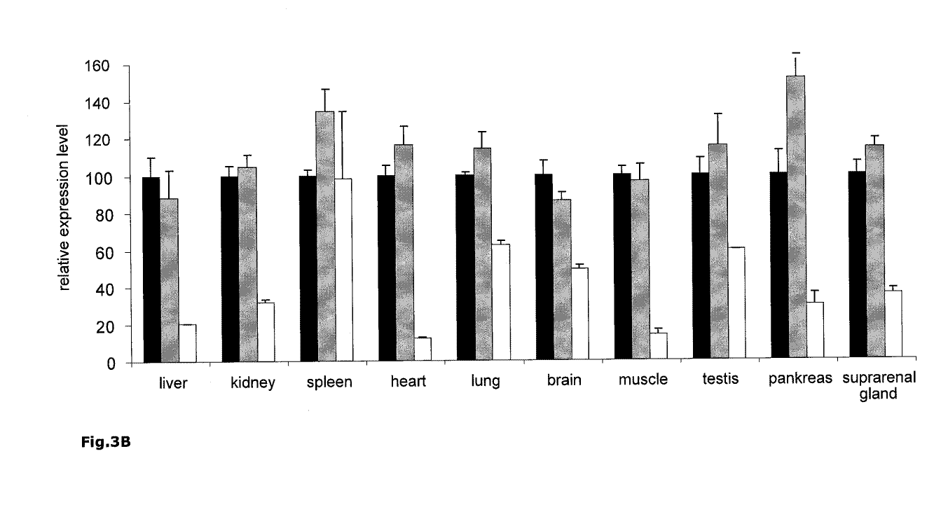

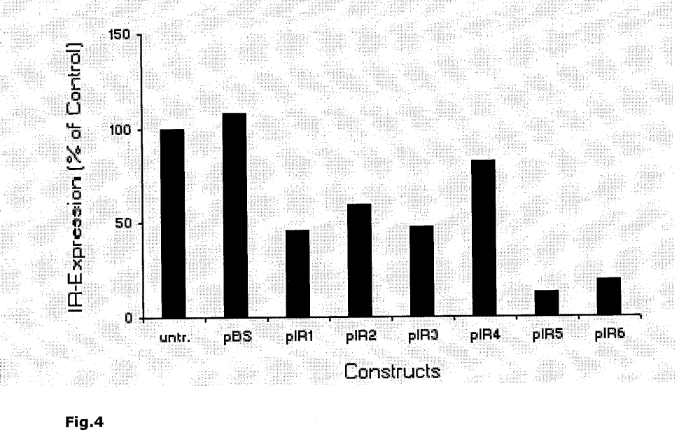

Mice were fed for 10 days with drinking water in the presence or absence of 2 μg/ml Doxycycline. FIG. 3 shows the firefly luciferase activity measured in different organs of mice. The Renilla luciferase gene at the second Rosa26 allele served as a reference to normalize the values of firefly luciferase activity. Doxycycline inducible expression of the shRNA under the control of the H1-tetO promoter (SEQ ID NO:3) resulted in a efficient reduction of firefly luciferase activity in most organs of mice expressing the wt tet repressor or expressing the codon optimized tet repressor (FIG. 3). Unexpectedly in the absence of doxycycline a efficient knockdown was measured for mice expressing the wt tet repressor (FIG. 3A; SEQ ID NOs:1 and 3). This demonstrates that the wt tet repressor is not able to inhibit the activation of H1-tetO driven shRNA through Polymerase III dependent promoter. In contrast, mice carrying the codon optimized tet repressor (FIG. 3B; SEQ ID NOs:2 and 3) did not show any detectable knockdown of luciferase in the absence of doxycycline. Moreover, the degree of RNAi upon induction was similar compared to the system using the wt repressor.

### Example 2

Vector construction: The following shRNA sequences were cloned 3′ of the H1-tet promoter (SEQ ID NO:222, nucleotides 158-391) followed by five thymidines.

IR1:

(SEQ ID NO:224)

agtccgcatcgagaagaatattcaagagatattcttctcgatgcggact
IR2:

(SEQ ID NO:225)

atcgagaagaataatgagctttcaagagaagctcattattcttctcgat
IR3:

(SEQ ID NO: 226)

actacattgtactgaacaattcaagagattgttcagtacaatgtagt
IR4:

(SEQ ID NO:227)

agggcaagaccaactgtcctttcaagagaaggacagttggtcttgccct
IR5:

(SEQ ID NO: 228)

agaccagacccgaagatttcttcaagagagaaatcttcgggtctggtct
IR6:

(SEQ ID NO:229)

agcctggctgccaccaatacttcaagagagtattggtggcagccaggct

The resulting vectors were named pIR1-pIR6. For example the sequence of pIR5 (SEQ ID NO:222) contains the shRNA IR5 (SEQ ID NO:222, nucleotides 393-440 and SEQ ID NO:228).

Rosa26/CAGGS-tetR/Insulin-receptor-shRNA exchange vector (FIG. 5): The vector contains the F3 site and the FRT site in the same configuration as in the rosa26 targeting vector described in Seibler et al., Nucleic Acids Res. 2005 Apr. 14; 33(7):e67 and PCT/EP05/053245. The pIR5-tet vector (SEQ ID NO:223) has the following order in 5′ to 3′ direction: synthetic polyA signal (SEQ ID NO:223, nucleotides 1-179), F3-site (SEQ ID NO:223, nucleotides 194-241), neomycin-resistance gene lacking the start ATG (SEQ ID NO:223, nucleotides 249-1046), PGK-pA site (SEQ ID NO:223, nucleotides 1072-1537), hgH polyA signal (SEQ ID NO:223, nucleotides 1565-2362), H1-tet promoter (SEQ ID NO:223, nucleotides 2538-2771), IR-5-specific shRNA sequence (SEQ ID NO:223, nucleotides 2773-2820), five thymidines, CAGGS promoter (Okabe, Fabs Letters 407:313-19 (1997); SEQ ID NO:223, nucleotides 2829-4672), codon optimized tet-repressor gene (SEQ ID NO:223, nucleotides 4730-5353), synthetic polyA signal (SEQ ID NO:223, nucleotides 5382-5560), FRT-site (SEQ ID NO:223, nucleotides 5576-5623).

Cell culture: Cultures of ES cells were carried out as described in Hogan, B. et al., A Laboratory Manual. In Manipulating the Mouse Embryo. Cold Spring Harbor Laboratory Press, Cold Spring Harbor N.Y., pp. 253-289 (1994) with ES cell lines derived from F1 embryos. Transfection of Art4.12 ES cells containing the FRT/F3 configuration with the pIR5-tet (SEQ ID NO:223) exchange vector has been described in Seibler et al., Nucleic Acids Res. 2005 Apr. 14; 33(7):e67 and PCT/EP05/053245.

Doxycycline induction of ES cells: Cells were treated with 1 μg/ml doxycycline (Doxycycline Hyclate, Sigma D-9891) for 48 h and medium was changed every day.

Transient transfections of muscle cells: C2C12 myoblasts were grown at 37° C. in an atmosphere of 5% CO₂ in Dulbecco's modified Eagle's medium (DMEM) containing 10% fetal calf serum (FCS), 4500 mg/l glucose and 1× non-essential amino acids. Transfection studies were carried out with 1.35×10⁵ cells plated on a 6-well plate. Cells were transfected 2.5 μg DNA (1.25 μg GFP-vector and 1.25 μg of one of the pIR1-6 vectors). DNA was mixed with 10 μl Lipofectamin (Invitrogen, #18324-111) and 200 μl Optimem (Gibco BRL, #51985-026) and incubated for 45 min at RT. For transfection, cells were washed with 1×PBS and incubated for 5 h in 2 ml starving medium, containing the Optimen-DNA-Solution. After 5 h medium DMEM with 20% FCS was added to the cells. 24 h after transfection cells were washed with 1×PBS and fixed with methanol for 3 min, washed with 1×PBS and dried. Cells were stained with DAPI in Vectashield (Vector). Cells were analyzed for GFP expression and transfection efficiency.

Mice: All mice were kept in the animal facility at Artemis Pharmaceuticals GmbH in micro-isolator cages (Tecniplast Sealsave). B6D2F1 Mice for the generation of tetraploid blastocysts were obtained from Harlan, N L.

Production of ES mice by tetraploid embryo complementation: The production of mice by tetraploid embryo complementation was essentially performed as described in Eggan et al., Proc Natl Acad Sci USA, 98, 6209-6214.

Doxycycline treatment: 2 mg/ml doxycycline (Doxycycline Hyclate, Sigma D-9891) was dissolved in water with 10% sucrose, 20 μg/ml doxycycline was dissolved in water with 1% sucrose and 2 μg/ml doxycycline was dissolved in water with 0.1% sucrose. The doxycycline solutions were freshly made every second day and kept dark.

Protein isolation: Cells were lysed in Protein extraction buffer containing 1% Triton® X-100, 0.1% SDS, 10 mM Tris-HCl pH 7.4, 1.25 mM Tris Base, 10 mM EDTA, 50 mM NaCl, 50 mM NaF, 50 μg Aprotinin protein concentration was measured using the Warburg formula.

Western Blot Proteins were fractionated on a 10% SDS-Page gel and semi-dry blotted for 30 min with 200 mA. Primary antibodies against Insulin receptor and AKT were from Santa Cruz and Cell Signaling Technology. IR antibody was diluted 1:200 and AKT 1:1000 in 2% milk powder (MP) in TBS. Second antibody was goat anti-rabbit IgG (whole molecule)-peroxidase (Sigma, #A6154-1mL), diluted 1:1000 in 2% MP/TBS used with ECL reagents (Amersham, #RPN 2105).

RNA isolation: Total RNA was isolated with peqGOLD TriFast (peqLab, #30-2020) using 2.5 ml for a confluent grown 10 cm plate. Cells were centrifuged for 15 min at 13000 rpm, 4° C. Supernatant was transferred in a new siliconized 2 ml Eppendorf tube and 0.3× volume Chloroform was added to the supernatant. The solution was mixed and centrifuged for 15 min at 13000 rpm, 4° C. The supernatant was transferred into a new siliconized 1.5 ml tube and was precipitated with the same volume of isopropanol. RNA was dissolved in DEPC-H₂O.

Northern Blot: 30 μg RNA were fractionated on a 15% denaturating polyacrylamid gel and blotted on a nylon membrane with an ampacity of 3.3 mA/cm² for 35 min. The RNA was cross-linked to the membrane using UV-light and incubation at 80° C. for 30 min. The membrane was incubated for 2 h in 10 ml prehybridisation solution and labeled with a radioactive probe specific for the used shRNA. 10 U T4-Polynukleotid-kinase (NEB) and 10 μCi γ-[³²P]-ATP (10 U μCi/μl) were used for labeling of the radioactive probe.

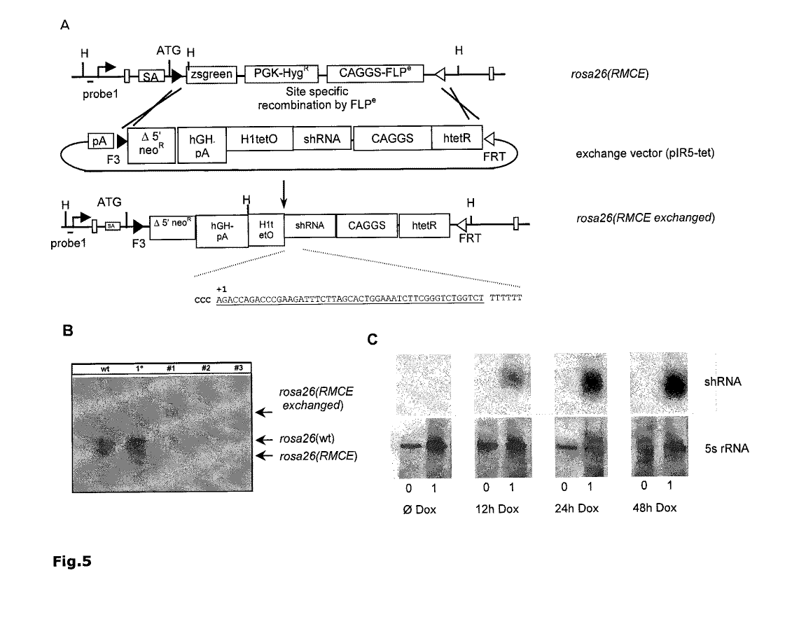

To investigate the potential of the Doxycycline (Dox) inducible shRNA expression system in vivo, the insulin receptor (IR) gene was chosen as a well-characterized target involved in glucose homeostasis and the development of Diabetes mellitus. Six different shRNA sequences directed against the IR mRNA (SEQ ID NO:221) were tested in the IR expressing muscle cell line C2C12. shRNA coding regions were cloned into a H1 expression vector (pIR1-6) and transiently transfected into C2C12 cells using lipofection. Western blot analysis of protein extracts derived from transfected cells revealed a significant RNAi activity of shRNA constructs pIR5 and pIR6, leading to a >80% reduction of IR expression (FIG. 4).

The RMCE strategy (Seibler et al., Nucleic Acids Res. 2005 Apr. 14; 33(7):e67) was subsequently used for targeted insertion shRNA sequence #IR-5 under the control of the H1tet promoter along with a constitutive expression cassette of the codon optimized tet-repressor (SEQ. ID NO:222; FIG. 5a). Upon transfection of embryonic stem (ES) cells, recombinase mediated integration of the exchange vector into the rosa26 locus was observed in >90% of G418 resistant colonies. Doxycyclin dependent expression in the resulting ES cell clones was assayed using Northern blot analysis, showing a high level of shRNA upon 12 h of induction with 1 μg/ml doxycycline (FIG. 5c).

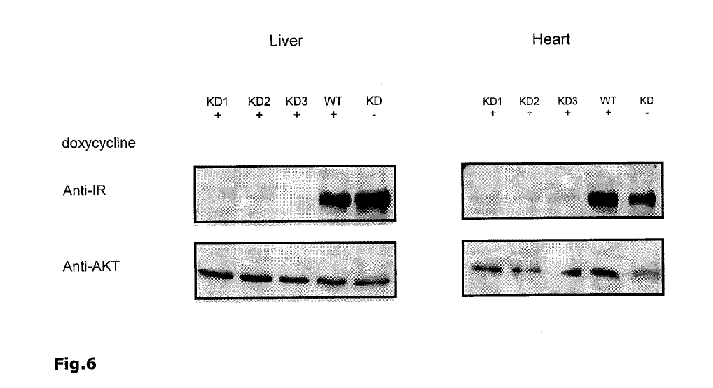

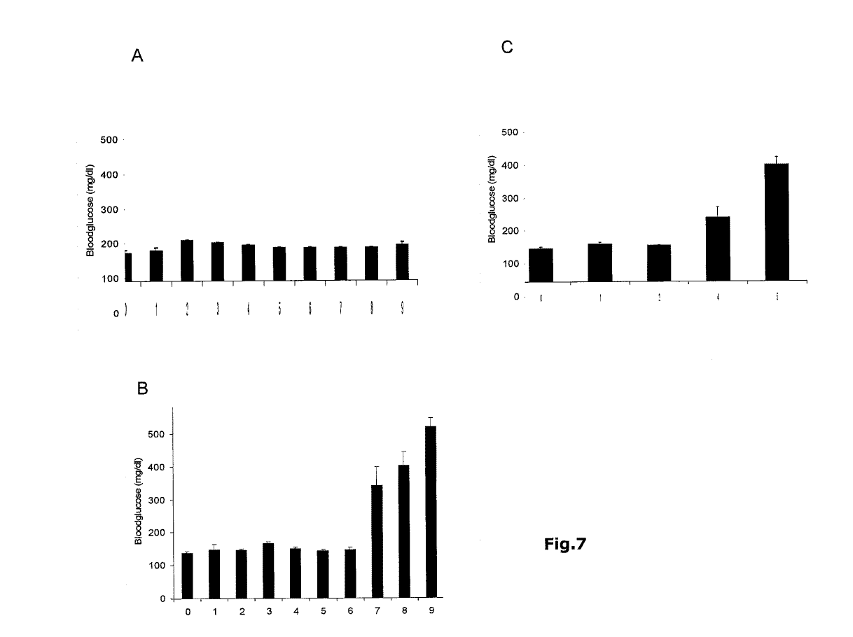

Mice were generated by injection of recombinant ES cell clones into tetraploid blastocysts (Eggan K. (2001) Proc Natl Acad Sci USA 98, 6209-6214.). Approximately six completely ES cell derived mice were obtained from 100 transferred blastocysts into pseudo-pregnant mothers. ShRNA transgenic mice were fed with 2 mg/ml doxycycline in the drinking water for 5 d and the degree of knockdown was detected at the protein level in liver and heart. Western blot analysis revealed a near complete removal of IR in Doxycycline treated animals, whereas the IR expression in untreated controls remained unaltered (FIG. 6).

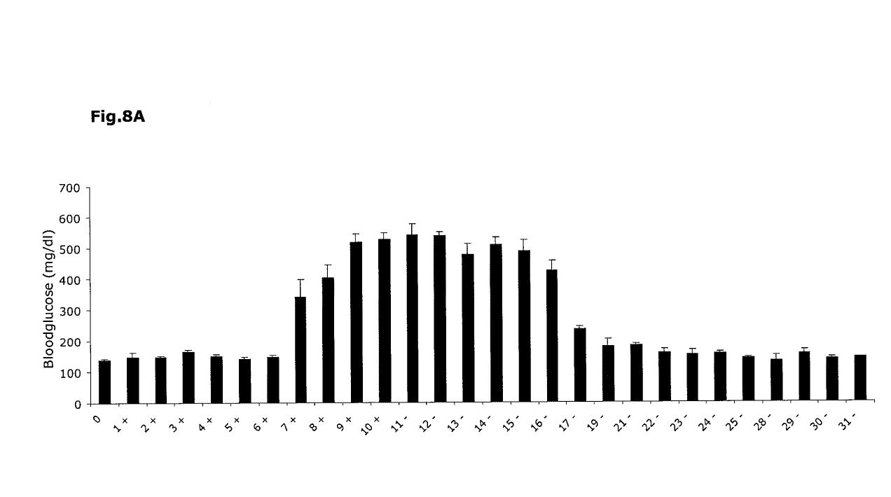

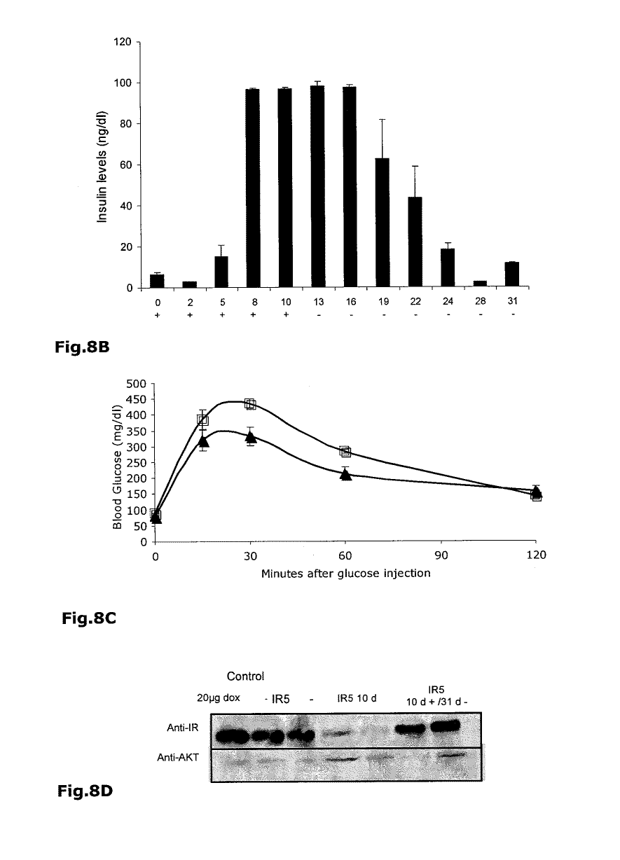

As a consequence of IR knockdown, Doxycycline-induced mice developed pronounced hyperglycemia. Blood glucose levels reached a maximum of ˜500 mg/dl at day 9 when treated with 20 μg/ml and at day 5 when treated with 2 mg/ml Doxycycline in the drinking water (FIG. 7). Upon withdrawal of 20 μg/ml Doxycycline serum glucose returned to normal levels within 7 d, demonstrating the reversibility of the Dox inducible promoter (FIG. 8). IR inducible knockdown mice did not show significant differences in glucose tolerance test before and after the induction of knockdown indicating a normal glucose metabolism after INSR knockdown (FIG. 8c). The reversible hyperglycemia is accompanied with a reversible knockdown of INSR in the liver as we detected the appearance of the protein after 21 days of the doxycycline removal (FIG. 8d).

### Example 3

### Comparative Example

Insertion of transgenes into the targeting vector: All subsequently described transgenes were inserted 3′ of the Renilla luciferase (Rluc) of the basic rosa26 targeting vector described in Example 1. The U6-promoter fragments were amplified from human genomic DNA (using the oligonucleotides ATCGGGATCCAGTGGAAAGAC GCGCAGG (SEQ ID NO:230) and GCTCTAGAAGACCACTTTCTCTATCACTGATAGGGAG ATATATAAAGCCAAGAAATCGA (SEQ ID NO:231)) and the tet-operator sequences was placed 3′ of the TATA-box resulting in the U6-promoter with the tet-operator sequence (U6-tet promoter; SEQ ID NO:232). 3′ of the U6-tet promoter a Fluc-specific shRNA was inserted by BbsI/XbaI using annealed oligonucleotides forming the sequence gggattccaattcagcgggagccacctgatgaagcttgatcgggtggctctcgctgagttggaatc cattttttt (SEQ ID NO:233; Paddison, P. J. et al., Genes Dev. 16:948-58 (2002)).

The resulting vector 4 (SEQ ID NO:234) contains the following elements in 5′ to 3′ orientation: 5′ homology region for rosa26 locus (nucleotides 25-1103), adenovirus splice acceptor site (nucleotides 1130-1250), Renilla luciferase (nucleotides 1326-2261), synthetic polyA (nucleotides 2279-2457), hgH-polyA (nucleotides 2490-3287), loxP-site (nucleotides 3308-3341), U6-tetO (nucleotides 3408-3671), shRNA (nucleotides 3672-3740), TTTTTT, loxP-site (nucleotides 3758-3791), FRT-site (nucleotides 3807-3854), PGK-hygro-polyA (nucleotides 3867-5676), FRT-site (nucleotides 5684-5731), 3′ homology region for rosa26 locus (nucleotides 5744-10075), PGK-Tk-polyA (nucleotides 10096-12280).

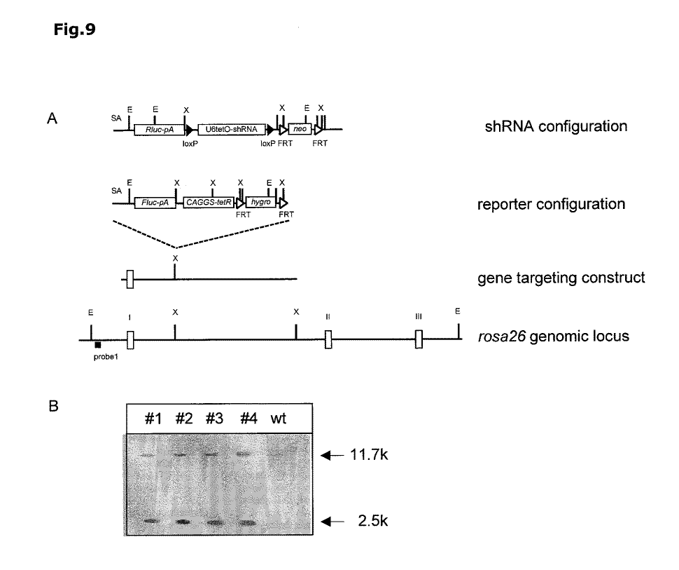

The U6-tet promoter construct (SEQ ID NO232) was tested using a dual reporter system consisting of firefly luciferase (Fluc) as a test substrate and Renilla reniformis luciferase (Rluc) as a reference (FIG. 9A). A firefly luciferase-specific shRNA sequence (SEQ ID NO 8) under the control of the U6-tet promoter along with the Renilla luciferase reporter construct (SEQ ID NO 234) and a wild type tetR gene along with a firefly luciferase reporter (SEQ ID NO 1) were introduced into the rosa26 locus through homologous recombination in embryonic stem (ES)-cells (FIG. 9A). Recombinant ES cells were identified through Southern blot analysis (FIG. 9B) and injected into blastocysts. Chimeric mice were obtained upon transfer of blastocysts into pseudo-pregnant females using standard protocols.

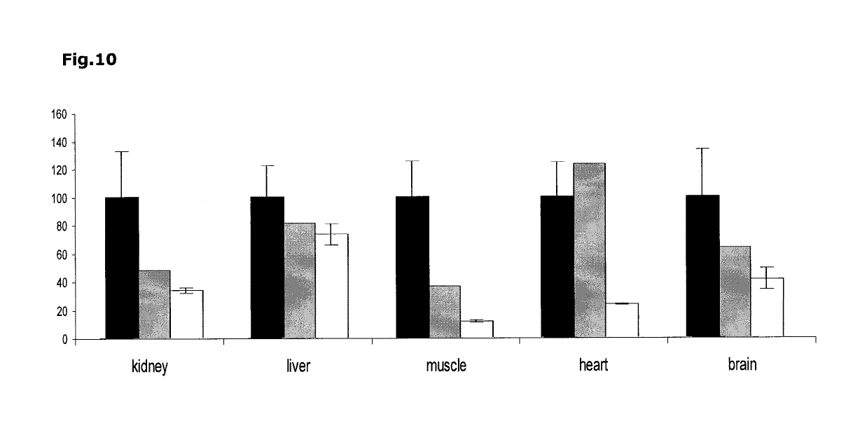

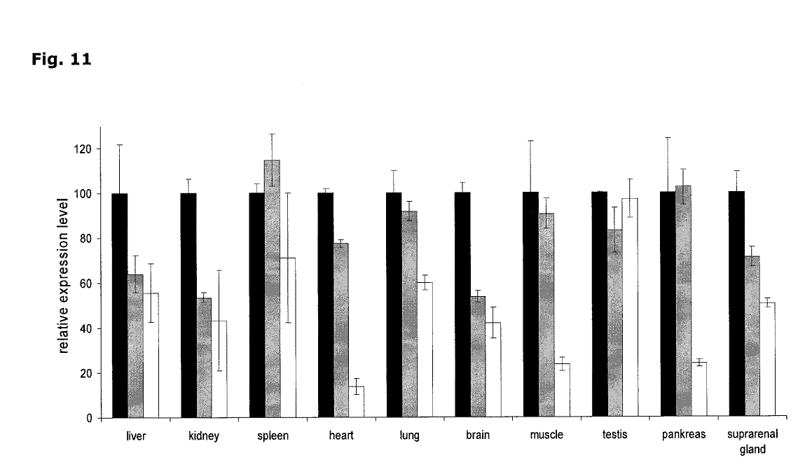

The relative firefly luciferase activity was determined in different organs of animals carrying the shRNA construct together with the luciferase- and tetR-transgenes. Upon induction with doxycycline, expression of the shRNA under the control of the engineered U6 promoter resulted in repression of firefly luciferase activity in most organs, ranging between 20-90% gene silencing (FIG. 10). A high degree background shRNA activity in the absence of doxycycline, particularly in kidney, muscle and brain was also detected (FIG. 10). In other organs such as liver and heart, leakiness seemed less pronounced, indicating that limited expression of tetR might be the reason for the incomplete block of RNAi in some tissues. A codon-optimized version of tetR (itetR, SEQ ID 2) was employed to improve regulation the shRNA constructs. ItetR was introduced into the Rosa26 locus in a similar configuration as the wild type tetR (FIG. 9A). The activity of firefly luciferase in the absence and in the presence of doxycycline was determined in different organs of the resulting mice. Again, the U6-tet promoter still showed residual activity in the absence of inductor (FIG. 11). This is in contrast to the data in WO 2004/056964, showing that a codon-optimized tetracycline repressor mediates tight regulation of a similar U6-tet promoter in cultured cell lines.

Sequence Listing Free Text

| | |
|---|---|
| SEQ ID NO: 1 | Targeting vector for rosa26 locus expressing |
| | the wt tet-repressor. |
| SEQ ID NO: 2 | Targeting vector for rosa26 locus expressing |
| | the codon optimized tet-repressor. |
| SEQ ID NO: 3 | Targeting vector for rosa26 locus containing |
| | the H1-tet inducible shRNA. |
| SEQ ID NOs: 4 and 5 | Primer Rscreen1s and Rscreen1as, respectively. |
| SEQ ID NO: 6 | 5′ arm for Rosa26 |
| SEQ ID NO: 7 | 3′ arm for Rosa26 |
| SEQ ID NO: 8 | firefly luciferase-specific shRNA. |
| SEQ ID NOs: 9 and 10 | Primer for isolation of codon optimized tet |
| | repressor |
| SEQ ID NO: 11 | Murine Rosa26 locus |
| SEQ ID NOs: 12 to 14 | siRNA sequences |
| SEQ ID NOs: 15 and 16 | Primer for isolation of SA from adenovirus |
| SEQ ID NO: 17 and 18 | Primer for isolation of H1 promoter |
| SEQ ID NOs: 19 to 220 | shRNA sequences, the function thereof being |
| | given in Tables 1 and 2 |
| SEQ ID NO: 221 | mouse insulin receptor (IR) mRNA |
| SEQ ID NO: 222 | vector pIR5 |
| SEQ ID NO: 223 | pIR5-tet vector |
| SEQ ID NOs: 224 to 229 | shRNA sequences IR1 to IR6 |
| SEQ ID NOs: 230 to 231 | Primer for isolation of U6 promoter with tet- |
| | operator |
| SEQ ID NO: 232 | U6-tet promoter |
| SEQ SEQ ID NO: 233 | firefly luciferase-specific shRNA in the U6-tet |
| | construct |
| SEQ SEQ ID NO: 234 | U6-tet targeting vector |

1. A biological entity selected from a non-human vertebrate, a tissue culture derived from a vertebrate or one or more cells of a cell culture derived from a vertebrate, said biological entity carrying
(i) a responder construct comprising at least one segment corresponding to a short hairpin RNA (shRNA) or to complementary short interfering RNA (siRNA) strands, said segment being under control of a ubiquitous promoter, wherein said promoter contains at least one operator sequence, by which said promoter is perfectly and ubiquitously regulatable by a repressor; and
(ii) a regulator construct comprising a codon-optimized repressor gene, which provides for perfect regulation of the promoter of the responder construct, wherein the responder construct and/or the regulator construct is (are) stably integrated into the genome of the biological entity, at a defined locus.

2. The biological entity according to claim 1, wherein
(i) said responder construct and said regulator construct allow inducible gene knock down in said biological entity, the regulation by said repressor permits control of the expression and the suppression of the expression of the shRNA or the siRNA by a rate of at least 90%; and/or
(ii) the responder construct and/or the regulator construct is (are) stably integrated into the genome of the biological entity, at a defined locus, by homologous recombination, recombinase mediated cassette exchange (RMCE) or the like; and/or
(iii) the responder construct and/or the regulator construct is (are) stably integrated, through homologous recombination or RMCE, at a defined genomic locus; and/or
(iv) the promoter of the responder construct is selected from polymerase (Poi) I, II and HI dependent promoters; and/or
(v) the promoter of the regulator construct is selected from polymerase (Pol) I, II and III dependent promoters; and/or
(vi) the responder construct and/or the regulator construct further contain functional sequences selected from splice acceptor sequences, polyadenylation sites, selectable marker sequences, recombinase recognition sequences; and/or
(vii) the responder construct and the regulator construct are integrated at the same locus or at different loci in the genome of the biological entity; and/or
(viii) the vertebrate is a non-human vertebrate.

3. The biological entity according to claim 1, wherein in the responder construct
(i) the promoter is a inducible promoter selected from polymerase (Pol) HI dependent promoters; and/or
(ii) the promoter contains an operator sequence selected from tetO, GaI0, lacO; and/or
(iii) the operator sequence of the promoter is positioned 1 to 10 bp 3′ (i.e., downstream) and/or 5′ (i.e., upstream) of the TATA element; and/or
(iv) the DNA sequence corresponding to the shRNA or siRNA is positioned 3′ to said operator sequence.

4. The biological entity according to claim 1, wherein the responder construct
(i) is integrated into a ubiquitously active Pol II dependent locus;
and/or
(ii) carries a Pol III dependent promoter containing the operator and the segment(s) corresponding to a shRNA or siRNA; and/or
(iii) comprises at least one shRNA segment having a DNA sequence A-B-C or C-B-A, or comprises at least two siRNA segments A and C or C and A, each of said at least two siRNA segments being under the control of a separate promoter, wherein
A is a 15 to 35 bp DNA sequence with at least 95% complementarily to the gene to be knocked down;
B is a spacer DNA sequence having 5 to 9 bp forming the loop of the expressed RNA hair pin molecule; and
C is a 15 to 35 bp DNA sequence with at least 85% complementarily to the sequence A; and/or
(iv) comprises a stop and/or a polyadenylation sequence.

5. The biological entity according to claim 1, wherein in the regulator construct
(i) the repressor gene is under control of an ubiquitous promoter; and/or
(ii) the repressor gene is a codon-optimized tet repressor, a codon-optimized Gal4 repressor, a codon-optimized lac repressor or a variant thereof.

6. The biological entity according to claim 1, wherein the biological entity is a mouse, mouse cell or mouse tissue, the responder construct comprises a H1-promoter sequence with one tet operator sequence positioned 1-2 bp 3′ of the TATA element and a DNA sequence encoding a shRNA lying 3′ to the said tet operator sequence, and the regulator construct comprises a codon-optimized tet repressor gene.

7. A method for preparing the biological entity as defined in claim 1, which method comprises stably integrating
(i) the responder construct, and
(ii) the regulator construct,
into the genome of the biological entity.

8. The method of claim 7
(i) which comprises subsequent or contemporary integration of the responder construct, and the regulator construct into the genome of vertebrate cells; and/or
(ii) wherein the integration of both, the responder construct and the regulator construct is effected by homologous recombination; and/or
(iii) wherein the integration of at least one of the responder construct and the regulator construct is effected by RMCE; and/or
(iv) wherein the integration is effected by using an integration vector carrying both, the responder construct and the regulator construct.

9. The method of claim 7, which is for preparing a transgenic nonhuman vertebrate and which comprises
(i) generating a first vertebrate or a first vertebrate line being transformed with the responder construct,
(ii) generating a second vertebrate or second vertebrate line being transformed with the regulator construct, and
(iii) crossing at least one of said first vertebrates with at least one of said second vertebrates.

10. Method of using a biological entity as defined in claim 1 for inducible gene knock down, and/or as a test system for pharmaceutical testing, and/or for gene target validation, and/or for gene function analysis.
